SEQUENCE LISTING (12 sequences)

SEQ ID NO: 1 - DNA, 22 bp, Artificial Sequence
  PCR primer
gagtggagaaagctgacctgtg

SEQ ID NO: 2 - DNA, 22 bp, Artificial Sequence
  PCR primer
cgccttctatcgccttcttgac

SEQ ID NO: 3 - DNA, 21 bp, Artificial Sequence
  PCR primer
cagtcatagccgaatagcctc

SEQ ID NO: 4 - DNA, 24 bp, Artificial Sequence
  PCR primer
agcagtccagaggctcacagcaac

SEQ ID NO: 5 - DNA, 20 bp, Artificial Sequence
  probe
ctagcatgatgagacaggtt

SEQ ID NO: 6 - DNA, 20 bp, Artificial Sequence
  probe
tccggtgcatgaagagatcc

SEQ ID NO: 7 - DNA, 21 bp, Artificial Sequence
  probe
cagcatgaatggccagagtcc

SEQ ID NO: 8 - DNA, 21 bp, Artificial Sequence
  probe
attggtccataacagcacatc

SEQ ID NO: 9 - protein, 42 aa, Mus musculus
DPTVFHKRYLKKIRDLGEGHFGKVSLYCYDPTNDGTGEMVAV

SEQ ID NO: 10 - protein, 42 aa, Mus musculus
DPTHFEKRFLKRIRDLGEGHFGKVELCRYDPEGDNTGEQVAV

SEQ ID NO: 11 - protein, 42 aa, Mus musculus
DPTQFEERHLKFLQQLGKGNFGSVEMCRYDPLQDNTGEVVAV

SEQ ID NO: 12 - protein, 42 aa, Mus musculus
DPAIFEERHLKYISLLGKGNFGSVELCRYDPLGDNTGPLVAV

(19) United States
(12) Patent Application Publication
(10) Pub. No.: US 2012/0034226 A1
(43) Pub. Date: Feb. 9, 2012

(54) DIAGNOSTIC METHOD OF SKIN INFLAMMATORY DISEASE

(75) Inventors: Hisahiro Yoshida, Yokohama-shi (JP); Takuwa Yasuda, Yokohama-shi (JP); Shigeharu Wakana, Tsukuba-shi (JP); Masato Kubo, Yokohama-shi (JP)

(73) Assignee: RIKEN, Wako-shi (JP)

(21) Appl. No.: 12/883,784

(22) Filed: Sep. 16, 2010

(30) Foreign Application Priority Data
Sep. 16, 2009 (JP) 215035/2009

(51) Int. Cl. A61K 39/395; C40B 30/00; A61P 29/00; C12Q 1/68; G01N 33/573; A61P 17/00; G01N 33/566

(52) U.S. Cl. 424/135.1; 435/7.92; 506/7; 435/7.21; 435/6.12

(57) ABSTRACT

The invention provides a method of determining (or diagnosing) the presence or absence of a functional abnormality in the skin barrier, a kit therefor, a method of ameliorating a functional abnormality in the skin barrier based on the onset mechanism, and a drug therefor. The method of determining the presence or absence of a functional abnormality in the skin barrier comprises measuring the expression of a protease and/or an inhibitor thereof on the skin surface of a test object, and the like.

### TECHNICAL FIELD

The present invention relates to a method of determining the presence or absence of a functional abnormality in the skin barrier, more specifically to a diagnostic method making it possible to diagnose at an earlier stage whether skin inflammation has developed or is likely to develop, by determining the presence or absence of a functional abnormality in the skin barrier, a kit therefor and the like. The present invention also relates to a method for ameliorating a functional abnormality in the skin barrier and a drug therefor and the like.

### BACKGROUND ART

Atopic dermatitis is a pruritic dermal syndrome found in 15% to 30% of children in developed countries, occurring in two types: cases thought to arise from an allergic predisposition and those thought to arise from an external factor not associated therewith. However, much remains unclear about the mechanism behind the onset thereof. In the former, individuals having a genetic disposition for a likely immunological accentuation of Th2 are prevalent; in fact, 70% of atopic dermatitis patients often exhibit a broad range of allergic diseases known as the allergic march or high serum IgE levels. On the other hand, 30% of atopic dermatitis patients do not exhibit an elevated Th2 or serum IgE level, and their conditions are not thought to have an immunological genetic disposition as the radical cause. The relatively prevalent occurrence in developed countries suggests the involvement of environmental factors such as hygiene and air pollution, and observed familial influences suggest the importance of hereditary factors. This disease is thought to develop as these factors act alone or in combination.

Meanwhile, prophylactic/therapeutic agents for atopic dermatitis that have been developed to date include immunosuppressant steroids, FK506 (tacrolimus), combinations thereof with moisture retention preparations, and the like, and various therapies using them have been reported as symptomatic treatments in view of clinical symptoms. Because the mechanism behind the development has not been clarified, however, it remains unknown whether these therapies are appropriately personalized, and as the situation stands, no effective treatments are available in terms of safety, efficacy, and psychological satisfaction due to some other problems, including the issue of adverse drug reactions in prolonged use and the spread of the sense of fear about steroid treatment stirred up by the media.

Additionally, because the pathogenetic mechanism is currently unknown, atopic dermatitis cannot be diagnosed and treated unless the illness has progressed to some extent, at which time a combination of coexisting infection and allergic disease makes the pathologic analysis complicated. This situation hampers the development of a therapeutic method; no reasonable therapy for theoretically preventing the onset has been developed.

Hence, there has been a demand for the provision of a method of predicting the onset of the illness (diagnostic method) based on the pathologic mechanism and a prophylactic/therapeutic agent for atopic dermatitis with an action mechanism based on the pathologic mechanism.

As experimental materials for research into atopic dermatitis, a plurality of mouse models of atopic dermatitis have been reported, which include five strains with spontaneous onset [NC/Nga, NOA, DS-Nh, MAD (see, non-patent document 1)), nad/nad (see, patent document 1)], seven strains generated by genetic engineering [IL-4 Tg, CASP1 Tg, IL-18 Tg, IL-31 Tg, Re1B-KO (see, non-patent document 1), CatE-KO (see, patent document 2 and non-patent document 1), MAIL-KO (see, patent document 3 and non-patent document 2)], four models of induced dermatitis with antigen application and the like, and those generated by transplanting blood components of an atopic dermatitis patient to a humanized mouse. Furthermore, an animal lacking the epiregulin gene is disclosed in patent document 4, and a GATA-3 gene-transfected animal model of atopic dermatitis in patent document 5. These models have their advantages and disadvantages and are subject to some limitations in use as mouse models of atopic dermatitis.

Meanwhile, the present inventors screened N-ethyl-N-nitrosourea (ENU)-induced mutant mice (hereinafter also simply referred to ENU mutant mice) to select an atopic dermatitis mouse, and reported on the phenotype thereof (patent document 6).

As far as the literature searched on the PubMed before Feb. 28, 2009, no reports have been presented suggestive of an association between mutations or activation of the Jak1 molecule in skin tissue and allergic diseases, including atopic dermatitis. Although a potential of a Jak inhibitor to the treatment of atopic dermatitis has been reported (patent document 7), the discussion is based on the involvement of a Jak molecule in immune responses, and does not demonstrate an association between the Jak1 molecule and atopic dermatitis not involved by immune responses.

A report is available that a signaling system containing the Jak1 molecule is associated with the maintenance of the homeostasis or the formation of a diseased state in the epidermis. It is described by non-patent document 3 that signaling by IL-6 and its receptors IL-6Rα and gp130 is important to the formation of the skin barrier. According to non-patent document 4, signaling by oncostatin M and its receptors OSMR and gp130 is important to the migration and differentiation of keratinocytes, and is involved in wound healing and inflammation. It is described by non-patent document 5 that activation of Stat3, a signaling factor downstream of the Jak1 molecule, is involved in skin carcinogenesis. It is stated by non-patent document 6 that activation and functional impairement of Socs3, an inhibitor of signaling by the Jak1 molecule, in the skin is associated with skin wound healing or carcinogenesis. However, none of these literature documents demonstrates an association between the Jak1 molecule and the development of atopic dermatitis.

Regarding Tyk2, which belongs to the same family as that of the Jak1 molecule, or the Stat3 molecule, which functions downstream thereof, a genetic mutation has been reported in human patients with congenital hyper-IgEemia which is a congenital human disease (non-patent documents 7 and 8). Although these patients have a similarity in the aspect of having atopic dermatitis-like symptoms, the Tyk2 or Stat3 mutation in these patients causes a primary immunodeficient state due to the inhibition of the function or differentiation of immunocytes, and does not contribute to the elucidation of the mechanism behind the onset of atopic dermatitis.

Regarding the onset of atopic dermatitis due to collapse of the barrier function of the skin, it has been shown that congenital ichthyosis or atopic dermatitis develops due to a mutation of the Filaggrin molecule (non-patent document 9), that a mutation for activation of KLK7, a protease required for the turnover of skin keratin, also known as SCCE, causes atopic dermatitis due to destruction of the skin barrier (non-patent document 10), and that conversely, a deletion mutation of the SPINK5 molecule, an inhibitory factor that suppresses the activation of KLK7, results in the onset of Netherton syndrome, a congenital disease, whose pathologic conditions include atopic dermatitis (non-patent document 11). Furthermore, a skin barrier function ameliorator (patent document 8) and a composition for moisture retention (patent document 9) have been reported.

Regarding the association between the accentuation of the expression of Chitinase 3 like 1 and the onset of inflammatory disease, accentuated expression in the airway epithelium of asthma patients and elevated concentrations in the serum have been reported (non-patent document 12), and an increased prevalence of a particular SNP of the same gene reported in a population of asthma patients has also been reported (non-patent document 13). Prior to these reports, it was reported that the expression of chitinase could be a target for the treatment of allergic diseases such as asthma (patent document 10). Also, accentuated expression is observed in the intestinal epithelium of patients with ulcerative colitis (non-patent document 14), and forced expression in intestinal epithelium allows the easier invasion of pathogenic bacteria (non-patent document 15). With these reports and the like, the association between Chitinase 3 like 1 and the pathologic conditions of allergic diseases and inflammatory diseases such as asthma and ulcerative colitis has already been pointed out (patent document 11).

Regarding the onset of atopic dermatitis, the concept of the development and progression of the illness has been presented by Bieber in the New England Journal of Medicine in 2008 (non-patent document 16), based on a bibliographic consideration.

In summary, it is described that atopic dermatitis has a time course wherein a trouble of skin component initially causes a barrier disorder, which is stimulated by allergens and adjuvants and then leads to the onset of allergic inflammation of the Th2 type, which is influenced by IgE, and the inflammation exacerbates due to scratching behavior and infections; this cycle occurs repeatedly and results in chronic inflammation.

However, no knowledge is available on details of the skin component changes that occur while inflammatory skin diseases such as atopic dermatitis develop from the symptom-free state and progress.
[patent document 1] JP-A-2004-105113
[patent document 2] JP-A-2004-041123
[patent document 3] JP-A-2004-321112
[patent document 4] JP-A-2004-290038
[patent document 5] JP-A-2004-166696
[patent document 6] JP-A-2007-075033
[patent document 7] JP-A-2009-508968
[patent document 8] JP-A-2008-001599
[patent document 9] JP-A-2008-001666
[patent document 10] JP-A-2006-508014
[patent document 11] JP-A-2008-546752
[non-patent document 1] Gutermuth J. et al., Mouse models of atopic eczema critically evaluated. Int Arch Allergy Immunol. 2004, 135, 262-276
[non-patent document 2] Shiina T. et al., Targeted disruption of MAIL, a nuclear I kappa B protein, leads to severe atopic dermatitis-like disease. J Biol Chem. 2004, 279, 55493-55498
[non-patent document 3] Wang X. P. et al., The interleukin-6 cytokine system regulates epidermal permeability barrier homeostasis. J Invest Dermatol. 2004, 123(1), 124-31
[non-patent document 4] Boniface K., et al., Oncostatin M secreted by skin infiltrating T lymphocytes is a potent keratinocyte activator involved in skin inflammation. J. Immunol. 2007, 178(7), 4615-22
[non-patent document 5] Kim D. J. et al., Constitutive activation and targeted disruption of signal transducer and activator of transcription 3 (Stat3) in mouse epidermis reveal its critical role in UVB-induced skin carcinogenesis. Oncogene 2009, 28(7), 950-60
[non-patent document 6] Zhu B. M., et al., Socs3 negatively regulates the gp130-Stat3 pathway in mouse skin wound healing. J Invest Dermatol. 2008, 128(7), 1821-9
[non-patent document 7] Minegishi Y. et al., Human tyrosine kinase 2 deficiency reveals its requisite roles in multiple cytokine signals involved in innate and acquired immunity. Immunity 2006, 25, 745-55
[non-patent document 8] Minegishi Y. et al., Dominant-negative mutations in the DNA-binding domain of Stat3 cause hyper-IgE syndrome. Nature 2007, 448, 1058-62
[non-patent document 9] Palmer C N. et al., Common loss-of-function variants of the epidermal barrier protein filaggrin are a major predisposing factor for atopic dermatitis. Nat. Genet. 2006, 38, 441-446
[non-patent document 10] Vasilopoulos Y. et al., Genetic association between an AACC insertion in the 3′UTR of the stratum keratin chymotryptic enzyme gene and atopic dermatitis. J Invest Dermatol. 2004, July; 123(1), 62-6
[non-patent document 11] Stoll C. et al., Severe hypernatremic dehydration in an infant with Netherton syndrome. Genet Couns. 2001, 12, 237-43
[non-patent document 12] Chupp G L. et al., A chitinase-like protein in the lung and circulation of patients with severe asthma. N Engl J. Med. 2007 Nov. 15; 357(20):2016-27
[non-patent document 13] Ober C. et al., Effect of variation in CHI3L1 on serum YKL-40 level, risk of asthma, and lung function. N Engl J. Med. 2008 Apr. 17; 358(16):1682-91. Epub 2008 Apr. 9
[non-patent document 14] Vind I. et al., Serum YKL-40, a potential new marker of disease activity in patients with inflammatory bowel disease. Scand J. Gastroenterol. 2003 June; 38(6):599-605
[non-patent document 15] Mizoguchi E., Chitinase 3-like-1 exacerbates intestinal inflammation by enhancing bacterial adhesion and invasion in colonic epithelial cells. Gastroenterology. 2006 February; 130(2):398-411
[non-patent document 16] Bieber T., Atopic dermatitis. N. Engl. J. Med. 2008, 358, 1483-94

### SUMMARY OF THE INVENTION

### Problems to Be Solved by the Invention

The present invention is intended to provide a method of determining (a method of diagnosing) the presence or absence of a functional abnormality in the skin barrier and a kit therefor. The present invention is also intended to provide a method of ameliorating a functional abnormality in the skin barrier on the basis of a mechanism of onset, and a drug therefor.

### Means of Solving the Problems

In view of the above-described problems, the present inventors conducted extensive investigations and identified the causes of onset of inflammatory skin diseases (e.g., atopic dermatitis) at the molecular level. The findings are summarized as follows:

1. In case of an inflammatory skin disease whose symptoms are not induced by an immunological abnormality, that is, in case of an inflammatory skin disease whose symptoms are due to a skin trouble such as destruction of the skin barrier (a functional abnormality in the skin barrier), the symptoms are caused by a change in the expression of a particular protease or an inhibitor thereof in skin tissue.
2. As a change in the expression of a particular protease or an inhibitor thereof, a (abnormal) functional accentuation of the Jak1-related intracellular signaling system is found.

Therefore, based on these findings, the present inventors found that:

i) it is possible to determine the presence or absence of a functional abnormality in the skin barrier by measuring a change in the expression of a particular protease or an inhibitor thereof in skin tissue, and
ii) it is possible to determine at which stage on the time course of inflammatory skin diseases such as atopic dermatitis the subject (test object) stands, according to the cause of onset, that is, when no elevation of serum IgE concentration is observed in the subject, it is possible to determine whether the subject stands at the initial stage of inflammatory skin disease on the basis of the presence or absence of a functional abnormality in the skin barrier, or to predict whether the subject will contract inflammatory skin disease, which resulted in the completion of the present invention.

Accordingly, the present invention provides:

[1] a method of determining the presence or absence of a functional abnormality in the skin barrier, comprising the step of measuring the expression of a protease and/or an inhibitor thereof on a skin surface in a test object,
[2] the method according to [1] above, wherein the method comprises the step of collecting serum from the test object and determining the IgE concentration in the serum, and further comprises the step of differentially diagnosing whether IgE mediates the functional abnormality in the skin barrier in the test object,
[3] the method according to [2] above, wherein the protease and/or inhibitor thereof is at least one kind selected from the group consisting of:

### Keratin 77;

Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity
(granulocyte-macrophage);
Interferon regulatory factor 7;
Ets homologous factor;
Leucine-rich alpha-2-glycoprotein 1;
SAM domain and HD domain, 1;

### Lipocalin 2;

Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and

### Keratin 16,

[4] the method according to [2] above, wherein the protease and/or inhibitor thereof is at least one kind selected from the group consisting of:
Predicted gene, OTTMUSG00000000971;
WAP four-disulfide core domain 12;
Kallikrein related-peptidase 6;
Extracellular proteinase inhibitor; and
Protease, serine 27,
[5] the method according to [2] above, wherein the method further comprises the step of measuring the presence or absence of a functional accentuation of the Jak1-related intracellular signaling system in the test object,
[6] the method according to [5] above, wherein the functional accentuation of the Jak1-related intracellular signaling system is an accentuation of Jak1 activity, an accentuation of Stat3 activity, and/or a reduction in Socs3 activity,
[7] the method according to [2] above, wherein the test object has developed or is likely to develop skin inflammation,
[8] the method according to [7] above, wherein the method comprises determining the presence or absence of a functional abnormality in the skin barrier in a test object differentially diagnosed to be not mediated by IgE in the step of measuring the IgE concentration in the serum,
[9] the method according to [8] above, wherein the method comprises using a skin tissue sample from the test object,
[10] a diagnostic kit for a functional abnormality in the skin barrier, comprising a substance capable of specifically detecting at least one kind selected from the group consisting of:

### Keratin 77;

Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;

### Lipocalin 2;

Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and

### Keratin 16,

[11] the kit according to [10] above, wherein the kit comprises a substance capable of specifically detecting at least one kind selected from the group consisting of:
Predicted gene, OTTMUSG00000000971;
WAP four-disulfide core domain 12;
Kallikrein related-peptidase 6;
Extracellular proteinase inhibitor; and
Protease, serine 27,
[12] the kit according to [11] above, wherein the kit further comprises a material having a level of adhesive force for collecting a skin tissue sample,
[13] a prophylactic and/or therapeutic drug for inflammatory skin disease comprising a substance that inhibits a functional accentuation of the Jak1-related intracellular signaling system, wherein the drug is to be applied to the skin,
[14] the drug according to [13] above, wherein the substance that inhibits a functional accentuation of the Jak1-related intracellular signaling system is a Jak1 inhibitor, Socs3, and/or a Stat3 inhibitor,
[15] a method of ameliorating a functional abnormality in the skin barrier, comprising (a) the step of measuring the expression of a protease and/or an inhibitor thereof on a skin surface in a test object, (b) the step of measuring a serum IgE concentration, and (c) the step of administering an effective amount of a substance that inhibits a functional accentuation of the Jak1-related intracellular signaling system to a test object that exhibited a change in the component balance of the protease and/or inhibitor thereof on the skin surface in the step (a), and did not exhibit a rise in the serum IgE concentration in the step (b),
[16] the method according to [15] above, wherein the protease and/or inhibitor thereof is at least one kind selected from the group consisting of:

### Keratin 77;

Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity (granulocyte-macrophage);
Interferon regulatory factor 7;
Ets homologous factor;

Leucine-rich alpha-2-glycoprotein 1;

SAM domain and HD domain, 1;

### Lipocalin 2;

Alcohol dehydrogenase 6A (class V);
Membrane-spanning 4-domains, subfamily A, member 6D;
soriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and

### Keratin 16,

[17] the method according to [15] above, wherein the protease and/or inhibitor thereof is at least one kind selected from the group consisting of:
predicted gene, OTTMUSG00000000971;
WAP four-disulfide core domain 12;
Kallikrein related-peptidase 6;
Extracellular proteinase inhibitor; and
Protease, serine 27,
[18] the method according to [15] above, wherein the test object has developed or is likely to develop skin inflammation,
[19] the method according to [15] above, wherein the skin inflammation is of the non-IgE-mediated type,
[20] the method according to [15] above, wherein a skin tissue sample from the test object is used in the step (a),
[21] the method according to [15] above, wherein the substance that inhibits a functional accentuation of the Jak1-related intracellular signaling system is a Jak1 inhibitor, Socs3, and/or a Stat3 inhibitor.

### Effect of the Invention

According to the present invention, it is possible to determine at which stage on the time course of inflammatory skin diseases such as atopic dermatitis the subject stands, according to the cause of onset. Hence, it is possible to diagnose an inflammatory skin disease prior to the onset of Th2 type-allergic inflammation, which is influenced by IgE, or to predict the development of an inflammatory skin disease in the subject. Identifying the cause of onset allows a therapeutic policy including a medication plan to be determined. Furthermore, according to the present invention, it is possible to diagnose inflammatory skin diseases such as atopic dermatitis conveniently and quickly.

### BRIEF DESCRIPTION OF THE DRAWINGS

FIG. 1 shows results of identification of a mutation of a causal gene in an atopic dermatitis mouse.

a: The position of the Jak1 gene on mouse chromosome 4.
b: A point mutation of the 2633rd nucleotide in the coding region of Jak1 (a mutation from guanine to adenine; G2633A) was observed in genome sequencing on a mouse having a mutant phenotype; this point mutation resulted in a change in the amino acid sequence of Jak1 from the 878th arginine to histidine (R878H).
c: The above-described amino acid substitution in Jak1 is present in the tyrosine kinase domain of Jak1 protein.

FIG. 2 shows a portion of the tyrosine kinase domain in the Jak family, wherein the enclosed regions are ATP-binding consensuses.

FIG. 3 shows evidence for a functional accentuation of Jak1 detected in a mouse embryonic fibroblast strain (MEF cells).

a: Results of measurements by immunoprecipitation of the phosphorylation of Jak1 and Stat3 in MEF cells established from a mutant mouse (homozygote; Mutant) and a wild-type mouse (WT); measured before and after stimulation with IL-6.
b: Results of measurements by immunoprecipitation of the phosphorylation of Jak1 and Stat3 in 293T cells expressing the G-CSFR-gp130 chimeric receptor, transfected with HA-tagged mutant Jak1 and wild-type Jak1.
c: Results of an examination of the proliferation potentials of CD4-positive lymphocytes obtained from the spleens of a mutant mouse (homozygote: mutant) and their litter mates (heterozygotes: hetero, wild-type mice: WT), which were stimulated with various cytokines. In response to IL-2, IL-4, IL-7, IL-6 and IFN-γ, the lymphocytes of the homozygote better proliferated than the lymphocytes of the heterozygotes and the wild-type mice. Stimulation with ionomycin and PMA also allowed the lymphocytes of the homozygote to better proliferate.
d: A graph showing that accentuated proliferation in response to IL-12 was not observed in the lymphocytes of any phenotype of mouse. Splenic CD4-positive lymphocytes of a mutant mouse (homozygote) and its litter mates (heterozygotes, wild-type mice) were stimulated with IL-12, which transmits stimulation via Jak2, not via Jak1. No difference in proliferation was noted among the litter mates. The lymphocytes derived from the mutant mouse better proliferated with PMA stimulation.
e: A graph showing the inhibitory effect of a pan-Jak-specific inhibitor (Jak inhibitor I; pyridone 6) on the accentuated proliferation of lymphocytes. The accentuated proliferation of the lymphocytes derived from the mutant mouse and the lymphocytes of other genotypes was inhibited dose-dependently. This demonstrates that the proliferation potential is induced by the Jak-mediated signaling system.
f: A graph showing the inhibitory effect of a Jak3-specific inhibitor (Jak 3 inhibitor I; WHI-P131) on the accentuation of the proliferation of lymphocytes. The lack of proliferation inhibition demonstrates that the proliferation is independent of the function of Jak3.

FIG. 4 shows the influences of a point mutation in Jak1 on the transcription levels of Jak1 and Stat family gene mRNAs. To examine the likelihood of activation of the Jak1 and Stat family protein at the transcription level, mRNA expression levels in various tissues (bone marrow, spleen, skin, thymus, lymph nodes and MEF cells) were examined. No significant difference was noted among the homozygote, heterozygote and wild-type.

FIG. 5 shows the effects of a Jak inhibitor on the development of dermatitis. The development of dermatitis was suppressed by applying the Jak inhibitor to the skin.

a: Evidence for intense accumulation of phosphorylated Jak1 protein (pY-Jak1) in epidermal layers affected by dermatitis.
b: A graph showing the influence of the Jak inhibitor on clinical symptoms in the ear skins of mutant mice. Non-treated mutant mice (-∘-; No application), mutant mice treated with a vehicle alone in the absence of the Jak inhibitor (--; DMSO/AOO), mutant mice treated with a vehicle in the presence of the Jak inhibitor (-▪-; Inhibitor/AOO).
c: Results of examinations of the statuses of expression of differentiation marker molecules (Ki67, K6, K14, K10) in the epidermal layer by histoimmunological staining. Examined were the expression of each differentiation marker molecule in wild-type mice (WT) and mutant mice (Mutant), and the expression of each differentiation marker molecule in mutant mice treated with the Jak inhibitor for 2 weeks from Week 4 of age to inhibit Jak1 activity. Control mice were treated with the vehicle alone in the absence of the Jak inhibitor.

FIG. 6 shows results of examinations of the destruction of skin barrier permeability in dermatitis mice.

a: Measurements of trans-epidermal water loss (TEWL) from the ear skins of mutant mouse homozygote and heterozygote, and wild-type mice.
b: Results of an examination of the destruction of the skin barrier in mutant mice using a phosphate-buffered solution (PBS) containing biotin particles.
c: A graph showing the influence of petrolatum coating on clinical symptoms in the ear skins of a mutant mouse. An untreated mutant mouse (-∘-; No application) and a mutant mouse receiving petrolatum coating on the skin (--; Petrolatum).

FIG. 7 shows results of a quantitative RT-PCR analysis in the skins of mutant mice. The expression of IL-4 increased in the mutant mice.

FIG. 8 shows the procedures for constructing a Jak1 mutant knock-in mouse.

FIG. 9 shows results of an examination of the expression of marapsin and chitinase 3-like 1 on the skin surfaces of a mutant mouse and a wild-type mouse.

### MODES FOR EMBODYING THE INVENTION

All technical terms and scientific words used herein have the same meanings as those that are generally understood by those skilled in the technical field to which the present invention belongs unless otherwise specified. Optionally chosen methods and materials that are identical or equivalent to those described herein can be used in embodying or testifying the present invention; preferred methods and materials are described below. The disclosures in all publications as well as patents, which are mentioned herein, are incorporated herein by reference for the purpose of, for example, describing and disclosing the constructs and methodologies that can be used in relation to the inventions described therein.

### (Method of Determining the Presence or Absence of a Functional Abnormality in the Skin Barrier)

The present invention provides a method of determining the presence or absence of a functional abnormality in the skin barrier, comprising the step of measuring the expression of a protease and/or an inhibitor thereof on a skin surface in a test object. The method further comprises the step of measuring serum IgE concentrations.

The protease and/or inhibitor thereof on a skin surface to be measured is not particularly limited, as far as its expression profile changes in the event of a functional abnormality in the skin barrier; specific examples include the following:

### Keratin 77;

Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;

### Lipocalin 2;

Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and

### Keratin 16.

These molecules are hereinafter also referred to as the molecule group-A.

The above-described series of molecules are such that the expression level has changed widely upon development of atopic dermatitis in an inflammatory skin disease, particularly in a mouse model of atopic dermatitis.

Molecules whose expression rises (upregulation) include:

Ubiquitin specific peptidase 18;
Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;

### Lipocalin 2;

Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
rPotease, serine 27 (marapsin);
Aldehyde oxidase 4;
Chitinase 3-like 1; and

### Keratin 16.

In mouse models of atopic dermatitis (e.g., Spade mouse), the expression of the above-described proteases and their inhibitor molecules in their skins begins to rise gradually before development of dermatitis.

Molecules whose expression weakens (downregulation) include:

### Keratin 77;

Hydroxyacyl-coenzyme A dehydrogenase; and
Betacellulin, epidermal growth factor family member.

The T-cell leukemia/lymphoma 1B, 3 molecule has the profile wherein the expression has weakened at 4 weeks after birth but increases at 6 weeks after birth.

By analyzing (measuring) the status of expression of at least one kind of molecule selected from among those predicted to be involved in the turnover of the keratinous portion of the epidermis, out of the above-described molecule group-A, i.e., Kallikrein related-peptidase 6, Predicted gene, OTTMUSG00000000971, WAP four-disulfide core domain 12, Extracellular proteinase inhibitor, Chitinase 3-like 1, and Protease, serine 27, it is possible to more effectively determine the presence or absence of a functional abnormality in the skin barrier.

To measure the expression of each of the above-described series of proteases and/or inhibitors thereof, a skin tissue sample from a test object is used. The skin tissue sample can be collected by a technique in common use in the art. A convenient method is such that a tape with a certain level of adhesive force or the like is plastered on a skin surface and kept in contact with the skin for a specified length of time, after which the tape is detached to collect a skin tissue sample. Various conditions such as adhesive force and the time of contact with the skin surface can be set as appropriate, as far as the desired amount of skin tissue is obtained.

The status of expression of the molecule can be analyzed (measured) using a method in common use in the art as it is or with an appropriate modification. Specifically, the analysis can be achieved by analyzing the status of expression using a substance possessing specific affinity for the molecule. Here, concerning the term “using”, the method is not particularly limited. For example, when the molecule is in the form of a gene, a method can be used wherein a hybridization reaction is utilized with the use of a substance possessing specific affinity for the gene. Substances possessing specific affinity for the gene include oligo- or polynucleotide probes (hereinafter also simply referred to as probes for the sake of convenience), and oligo- or polynucleotide primer pairs (hereinafter also simply referred to as primer pairs for the sake of convenience) which possess specific affinity for the gene. The specific affinity means the property of hybridizing to the desired gene only; therefore, the substance may be completely complementary to all or part of the gene, or may contain one to several mismatches, as far as the above-described features are ensured. The probe and primer pair are not particularly limited, as long as they possess specific affinity for the gene; examples include oligo- or polynucleotides comprising all or part of the base sequence of the gene or a sequence complementary thereto and the like, and they are chosen as appropriate according to the form of the gene to be detected. The oligo- or polynucleotide is not subject to limitations with respect to the derivation thereof, as far as it possesses specific affinity for the gene, and it may be a synthetic product and may be purified by a conventional method from the necessary portion cleaved out from the gene. These oligo- or polynucleotides may be labeled with a fluorescent substance, an enzyme, a radioisotope or the like.

Provided that only one kind of gene is targeted, it may alone be subjected to a hybridization reaction. When analyzing the status of expression of a plurality of kinds (preferably a plurality of kinds are analyzed) at one time, however, it is preferable to perform a microarray analysis. The microarray analysis can be performed using a method in common use in the art as it is or with an appropriate modification. For the sake of convenience, a commercial kit can be used as appropriate with substances possessing specific affinity for the target genes provided at hand.

When the molecule is in the form of a protein, for example, a method can be used wherein a substance possessing specific affinity for the protein is used.

Substances possessing specific affinity for the protein include, for example, an antibody possessing specific affinity for the protein or a fragment thereof, the specific affinity being the capability of specifically recognizing the protein and binding thereto by an antigen-antibody reaction. The antibody or fragment thereof is not particularly limited, as far as it is capable of specifically binding to the protein; it may be any one of a polyclonal antibody, a monoclonal antibody and a functional fragment thereof. These antibodies and functional fragments thereof are prepared by a method in common use in the art. For example, when using a polyclonal antibody, a method is available wherein an animal such as a mouse or a rabbit is immunized with the protein injected by subcutaneous administration in the back or by intraperitoneal or intravenous injection and the like, and an elevation of antibody titer is waited, after which antiserum is collected. When using a monoclonal antibody, a method is available wherein a hybridoma is generated by a conventional method and the secretion therefrom is collected. To produce an antibody fragment, a method is commonly used wherein a cloned antibody gene fragment is allowed to be expressed by a microorganism and the like. The purity of the antibody, antibody fragment and the like is not particularly limited, as far as specific affinity for the protein is retained. These antibodies or fragments thereof may be labeled with a substance capable of labeling, such as a fluorescent substance, an enzyme, or a radioisotope.

Furthermore, commercial products may be used.

Furthermore, the method of the present invention preferably further comprises the step of measuring the serum IgE concentration in the test object. By carrying out this step, it is possible to obtain a good hint to determine the skin status of the test object while with no elevation of serum IgE concentration, that is, whether the test object stands at a stage wherein a functional abnormality in the skin barrier has occurred but skin inflammation has not yet developed, or at an initial stage wherein inflammatory skin disease has developed but is not mediated by IgE. Thus, a treatment that is more suited for the onset mechanism can be started earlier.

A case is also likely wherein the test object has an inflammatory skin disease due to an immunological abnormality or an IgE-mediated inflammatory skin disease, but exhibits a transiently reduced serum IgE concentration due to a treatment such as medication. Therefore, for patients who have already started some treatment, it is preferable that the determination of serum IgE concentration be repeated several times, preferably at intervals of 4 to 6 weeks, before and after the start of treatment.

The step of collecting serum and measuring the IgE concentration in the serum can be carried out by a method in common use in the art or a method based thereon.

The method of the present invention may further comprise the step of measuring the presence or absence of a functional accentuation of the Jak1-related intracellular signaling system in the test object. The conduct of this step is based on the association between non-IgE-mediated skin inflammation or a functional abnormality in the skin barrier and a functional accentuation of the Jak1-related intracellular signaling system.

The Jak (Janus kinase) family plays a role in the cytokine-dependent control of the functions of cells involved in the proliferation and immune responses. Currently, four mammalian Jak family members are known to exist (Jak1, Jak2, Jak3, and Tyk2). Jak proteins have molecular weights of 120 to 140 kDa, comprising some conserved Jak homology (JH) domains. A tyrosine kinase domain is known to exist as a JH domain.

In the present invention, “the Jak1-related intracellular signaling system” is understood to mean a cascade that represents a signaling pathway mediated directly and indirectly by Jak1, and that comprises the step of allowing a cytokine of the IL-6 family or a growth factor such as EGF to bind to a receptor thereof to activate Jak, and the activated Jak to phosphorylate the tyrosine 705 of Stat3 to activate Stat3. Therefore, in addition to the Jak1 molecule, other molecules that mediate this pathway, that is, signaling molecules located upstream or downstream of the Jak1 molecule and signaling inhibitor molecules (hereinafter also generically referred to as Jak1-related molecules) are also included in “the Jak1-related intracellular signaling system”.

Jak1-related molecules include, for example, cytokines of the gp130-sharing group such as IL-6, IL-11, oncostatin M, LIF, CNTF, IL-27, and IL-31;
growth factors with a tyrosine kinase receptor, such as EGF, PDGF, and M-CSF;
cytokines of the type II cytokine receptor family such as IFN-γ, IFN-α/β/ω, IL-10, IL-19, IL-20, IL-22, IL-28, and IL-29;
cytokines that share γc receptor, such as IL-4, IL-13, IL-2, IL-7, IL-9, IL-15, and IL-21;
receptors related to the gp130 family such as IL6Rα, gp130. WSX1, OSMR, and IL31Rα;
tyrosine kinase receptors such as EGFR, PDGFRα, PDGFRβ, and c-fms;
members of the type II cytokine receptor family such as IFNGR1, IFNGR2, IFNAR1, IFNAR2, IL-28Rα, IL-10Rβ, IL-20Rα, and IL-22R;
upstream signaling molecules comprising γc receptors and receptors that form hetero-dimers therewith;
signaling molecules downstream of Jak1 molecule, such as Stat1, Stat3, and Stat5; and
Jak1 signaling inhibitor molecules such as Socs3;
and the like.

Hence, “the step of measuring the presence or absence of a functional accentuation of the Jak1-related intracellular signaling system” is a step for measuring the presence or absence of an (abnormal) accentuation of the expression or functional accentuation of the signaling system, caused by a mutation of the above-described Jak1-related molecules and the like, and can be carried out by, for example, measuring whether an accentuation of Jak1 activity, an accentuation of Stat3 activity or a reduction of Socs3 activity is noted. An accentuation of Jak1 activity can be evaluated by measuring the amount of the Jak1 molecule expressed or the amount of the Jak1 molecule phosphorylated by the self-phosphorylation potential of the Jak1 molecule, or the potential of the Jak1 molecule for phosphorylating Stat3. An accentuation of Stat3 activity can be determined by measuring the amount of the Stat3 molecule expressed or the amount of the Stat3 molecule phosphorylated. A reduction in Socs3 activity can be determined by measuring the amount of the Stat3 molecule expressed.

The amount of each molecule or the amount expressed thereof, including phosphorylated molecules, can be measured using a method in common use in the art as it is or with an appropriate modification; useful methods include a Western blotting method using specific antibodies against respective molecules and the like.

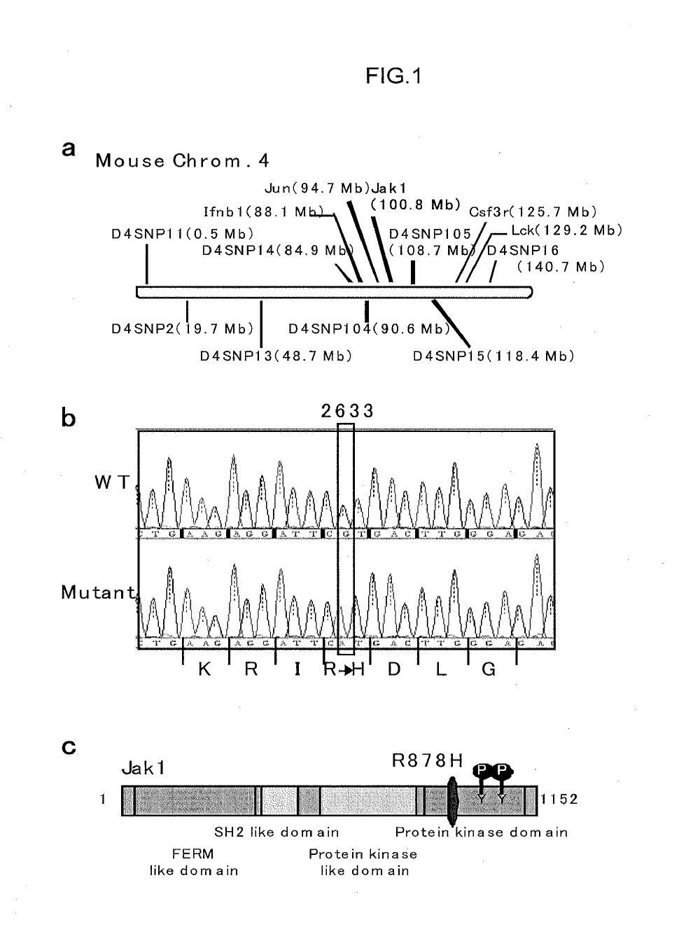

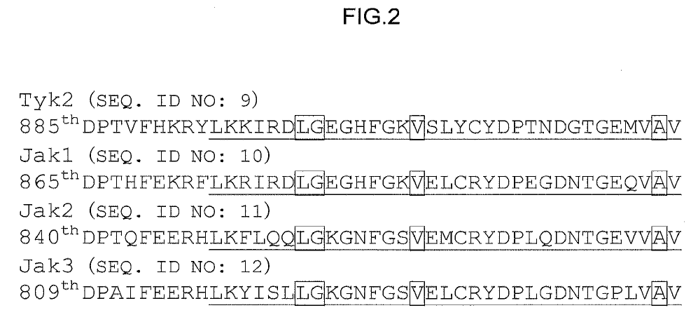

As described in detail in Examples, the present inventors found that a functional accentuation of the Jak1-related intracellular signaling system, which can be a cause of the development of an intrinsic inflammatory skin disease, can arise due to a mutation in a Jak1-related molecule, particularly the Jak1 molecule. In mice, for example, as a result of a point mutation of the 2633rd guanine to adenine in the coding region of the Jak1 gene [this point mutation results in a change in the 878th amino acid from arginine to histidine in Jak1 protein (see FIG. 1)], the phosphorylation potential of Jak1 is accentuated, that is, a functional accentuation of the Jak1-related intracellular signaling system is caused. This mutation in Jak1 occurs in the tyrosine kinase domain, representing a mutation within the consensus amino acid sequence shared by the Jak family, which consists of 32 amino acids from the 865th amino acid (aspartic acid) to the 906th in Jak1 protein (FIG. 2). Furthermore, this amino acid sequence of 32 amino acids is exactly the same in humans and mice both for Tyk2 and Jak1, representing a sequence conserved beyond the barrier of species. Furthermore, the amino acids from the 16th position in this sequence constitute the sequence of the ATP-binding domain.

The test object to which the diagnostic method of the present invention is applied is not particularly limited, as far as it is desired to be determined for the presence or absence of a functional abnormality in the skin barrier, or to be diagnosed with skin inflammation that has already developed or is likely to develop; examples include mammals, preferably mice, rats, other rodents, rabbits, dogs, cats, pigs, bovines, sheep, horses and primates, with greatest preference given to humans. In particular, the present invention allows a differential diagnosis to be made on the basis of the cause of the development of inflammatory skin disease, thus making it possible to diagnose an inflammatory skin disease not due to an immunological abnormality (non-IgE-mediated type); therefore, even in the absence of an elevation of serum IgE concentration, a treatment can be started as required. Therefore, the test object to be subjected to the diagnostic method of the present invention is suitably a test object that exhibits no elevation of serum IgE concentration.

The step of measuring the presence or absence of a functional accentuation of the Jak1-related intracellular signaling system using a sample collected from the above-described test object is hereinafter described specifically.

The choice of the sample is not particularly limited, as far as it allows the presence or absence of a functional accentuation of the Jak1-related intracellular signaling system to be measured. Because a functional accentuation of the intracellular signaling system is to be measured, however, the sample is preferably a genome sample from the test object. The genome sample may be a biological sample collected from the test object, or may be a genome solution extracted and purified from a biological sample and the like. Preference is given to biological samples of human derivation for use in clinical testing and the like, and genome solutions extracted and purified from biological samples of human derivation. Biological samples of human derivation include, for example, blood, spinal fluid, lymph fluid, urine, sputum, ascites fluid, exudates, amniotic fluid, intestinal lavage fluid, pulmonary lavage fluid, bronchial lavage fluid, vesical lavage fluid, pancreatic juice, saliva, semen, bile, feces and the like. The biological sample may be a sample in a state collected from an organism (e.g., biopsy sample), or may be a prepared sample. The method of preparation is not particularly limited, as far as the genome contained in the biological sample is not damaged; a method of preparation in common use for a biological sample can be used. Alternatively, the sample may be a genome solution extracted and purified from a biological sample, and amplified by PCR and the like. Another preferably used biological sample is a skin tissue sample. Use of a skin tissue sample is beneficial because it allows the status of expression of the above-described series of proteases and inhibitors thereof to be measured at one time. The biological sample may contain an inflamed portion or not. Furthermore, by using cord blood, placenta appendix and the like as the biological sample, it is possible to predict the development of inflammatory skin disease as early as at the time of birth.

The presence or absence of a functional accentuation of the Jak1-related intracellular signaling system is normally determined by comparing a measurement in a sample from a test object and a measurement in a control sample.

Here, regarding the use of “a control sample”, three cases are likely:

1. Use of a sample collected from a healthy individual that exhibits neither a functional abnormality in the skin barrier nor an inflammatory skin disease.
2. Use of a sample collected from a non-inflamed portion of an individual with a functional abnormality in the skin barrier or with an inflammatory skin disease.
3. Use of a sample collected from a previously inflamed portion of an individual with a functional abnormality in the skin barrier or with a skin inflammatory disease while the inflammation has soothed.

In predicting whether a certain individual will experience an inflammatory skin disease in the future, or in diagnosing the cause of development of a developed inflammatory skin disease, the control is a sample collected from a healthy individual as described in term 1 above. If there is a distinct difference in symptoms between the inflamed portion and the non-inflamed portion in an individual with an inflammatory skin disease, a set of genes with an accentuated function or expression due to site-specific activation can be identified by using a sample as described in term 2 above. If recurrence is suspected in an individual with soothed skin inflammation as judged by various therapeutic methods, the mechanism behind the inflammation relapse can be diagnosed by using a sample at the inflammation soothing stage as described in term 3 above as a control.

The inflammatory skin disease that can be diagnosed or predicted to develop by the method of the present invention is not particularly limited, as far as it is a disease accompanied by skin inflammation; specific examples include psoriasis, psoriasis guttata, inverse psoriasis, pustular psoriasis, psoriatic erythroderma, acute febrile neutrophilic dermatosis, eczema, xerotic eczema, dyshidrotic eczema, vesicular palmar eczema, acne vulgaris, atopic dermatitis, contact dermatitis, allergic contact dermatitis, dermatomyositis, exfoliative dermatitis, hand eczema, pompholyx, rosacea, rosacea due to sarcoidosis, rosacea due to scleroderma, rosacea due to Sweet syndrome, rosacea due to systemic erythematosus, rosacea due to urticaria, rosacea due to herpetic pain, Sweet's disease, neutrophilic hydrodenitis, sterile pustule, drug rash, seborrheic dermatitis, pityriasis rosea, Kikuchi's disease of the skin, pruritic urticarial papules and plaques of pregnancy, Stevens-Johnson syndrome and toxic epidermal necrolysis, tattoo reaction, Wells syndrome (eosinophilic cellulitis), reactive arthritis (Reiter syndrome), bowel-associated dermatosis-arthritis syndrome, rheumatoidneutrophilic dermatosis, neutrophilic eccrine hidradenitis, neutrophilic skin disease of dorsum of hand, balanitis circumscripta plasmacellularis, balanoposthitis, Behget's disease, erythema annulare centrifugum, erythema dyschromicum perstans, erythema multiforme, granuloma annulare, dermatitis of hand, lichen nitidus, lichen planus, lichen sclerosus et atrophicus, lichen simplex chronicus, lichen spinulosus, nummular dermatitis, pyoderma gangrenosum, sarcoidosis, subkeratinous pustular dermatosis, urticaria, transient acantholytic dermatosis and the like, with preference given to atopic dermatitis.

The inflammatory skin disease is preferably an inflammatory disease accompanied by destruction of the skin barrier. The barrier function of the skin is accounted for mainly by the keratinous layer (horny layer lipids, mainly ceramides, and natural moisture retention factors) and the sebum film that covers the surface thereof; as they work normally, the loss of water from the body is prevented to protect the skin against various stimuli from outside and the entry of foreign substances. Destruction of the skin barrier can be quantitatively judged using a method in common use in the art as it is or with an appropriate modification. Such methods include, for example, the riboflavin method and the method involving a measurement of trans-epidermal water loss (TEWL) and the like. The riboflavin method is such that skin barrier destruction is evaluated by transdermally smearing riboflavin, a low-molecular fluorescent substance, and measuring the amount penetrating the keratinous layer by the fluorescence intensity. TEWL can conveniently be measured under given conditions using a dedicated instrument.

When the function of the Jak1-related intracellular signaling system is accentuated, for example, when an accentuation of Jak1 activity, an accentuation of Stat3 activity or a reduction in Socs3 activity is noted, the test object is diagnosed as having contracted or possibly contracting an inflammatory skin disease due to a functional abnormality in the skin barrier, or is diagnosed as having contracted or possibly contracting a non-IgE-mediated inflammatory skin disease. Meanwhile, when skin inflammation has developed but the Jak1-related intracellular signaling system is not functionally accentuated, for example, when an accentuation of Jak1 activity, an accentuation of Stat3 activity or a reduction in Socs3 activity is not noted, the inflammatory skin disease is diagnosed as resulting from an immunological abnormality, or as being of the IgE-mediated type. Accordingly, the diagnostic method of the present invention provides a method of making a differential diagnosis of an inflammatory skin disease according to the cause of development.

### (Prophylactic/Therapeutic Drugs for Inflammatory Skin Disease)

The present invention provides a prophylactic and/or therapeutic drug for inflammatory skin disease comprising a substance that inhibits a functional accentuation of the Jak1-related intracellular signaling system (hereinafter also simply referred to as the active ingredient of the present invention). The drug is intended for skin application.

As is evident from the foregoing description of the method of the present invention and the Examples given below, a functional accentuation of the Jak1-related intracellular signaling system leads to the diagnostic judgement that the test object (subject) has contracted or is likely to contract an inflammatory skin disease due to a functional abnormality in the skin barrier, or has contracted or is likely to contract a non-IgE-mediated inflammatory skin disease. Therefore, a substance that inhibits a functional accentuation of the Jak1-related intracellular signaling system can be contained as an active ingredient in a prophylactic/therapeutic drug for an inflammatory skin disease, particularly an inflammatory skin disease due to a functional abnormality in the skin barrier, or a non-IgE-mediated inflammatory skin disease.

“A substance that inhibits a functional accentuation of the Jak1-related intracellular signaling system” is a substance capable of acting on Jak1 and/or a Jak1-related molecule (e.g., Socs3, Stat3) to inhibit a functional accentuation of the Jak1-related intracellular signaling system, and is exemplified by Jak1 inhibitors, inhibitors of Jak1 signaling molecules (e.g., Stat3), Jak1 signaling inhibitor molecules (e.g., Socs3) and the like. The functional accentuation may be one resulting from a mutation in Jak1 or a Jak1-related molecule. In this case, the substance that inhibits a functional accentuation is capable of acting on the mutant Jak1 and/or mutant Jak1-related molecule to inhibit a functional accentuation of the Jak1-related intracellular signaling system.

The Jak1 inhibitor, inhibitor of Jak1 signaling molecule and Jak1 signaling inhibitor molecule may be any commonly known compound or a novel compound; examples include nucleic acids (e.g., nucleosides, oligonucleotides, polynucleotides), sugars (e.g., monosaccharides, disaccharides, oligosaccharides, polysaccharides), lipids (e.g., fatty acids containing a saturated or unsaturated linear or branched chain and/or ring), amino acids, proteins (e.g., oligopeptides, polypeptides), organic low molecular compounds, compound libraries prepared using combinatorial chemistry technology, random peptide libraries prepared by solid phase synthesis or the phage display method, or naturally occurring ingredients derived from microorganisms, animals, plants, marine organisms and the like, and the like.

Described below are substances that can be included as “a substance that inhibits a functional accentuation of the Jak1-related intracellular signaling system” in the prophylactic and/or therapeutic drug for inflammatory skin disease of the present invention.

A nucleic acid having a nucleotide sequence complementary to a nucleotide sequence that encodes Jak1 or a Jak1 signaling molecule or a portion thereof (hereinafter also referred to as an “antisense nucleic acid”) can be designed and synthesized on the basis of information on the cloned or determined nucleotide sequence that encodes Jak1 or a Jak1 signaling molecule. Such a nucleic acid is capable of inhibiting the replication or expression of the Jak1 or Jak1 signaling molecule gene. Hence, the antisense nucleic acid is capable of hybridizing to the RNA transcribed from the gene that encodes the Jak1 or a Jak1 signaling molecule, thus inhibiting the synthesis (processing) or function (translation into protein) of mRNA.

The target region of the antisense nucleic acid is not subject to limitation as to the length thereof, as far as hybridization of the antisense nucleic acid results in the inhibition of the translation of the Jak1 or Jak1 signaling molecule, and the target region can be the entire sequence or a partial sequence of the mRNA or initial transcription product that encodes the protein; a partial sequence of about bases for the shortest, and the entire sequence of the mRNA or initial transcription product for the longest, can be mentioned. Considering the ease of synthesis and the issue of antigenicity, an oligonucleotide consisting of about 15 to about 30 bases is preferred, which, however, is not to be construed as limiting. Specifically, for example, the 5′-end hairpin loop, the 5′-end 6-base-pair repeat, the 5′-end untranslated region, the translation initiation codon, the protein-coding region, the translation stop codon, the 3′-end untranslated region, the 3′-end palindrome region, and the 3′-end hairpin loop of a nucleic acid that encodes the Jak1 or Jak1 signaling molecule (e.g., mRNA or initial transcription product) can be selected as the target region, but any region within the gene that encodes the Jak1 or Jak1 signaling molecule can be selected as the target.

Furthermore, the antisense nucleic acid may be not only one that hybridizes with the mRNA or initial transcription product that encodes the Jak1 or Jak1 signaling molecule to inhibit the translation into a polypeptide, but also one capable of binding to a double-stranded DNA that encodes the Jak1 or Jak1 signaling molecule to form a triple strand (triplex) and inhibit the transcription of RNA.

The choice of antisense nucleic acid may be a DNA or an RNA, or a DNA/RNA chimera. Because natural-type antisense nucleic acids have the phosphoric acid diester linkages thereof decomposed readily by nucleases being present in the cells, the antisense nucleic acid used in the present invention can also be synthesized using a modified nucleotide such as the thiophosphate type (phosphoric acid bond P═O replaced with P═S) or the 2′-O-methyl type, which are stable to nucleases. Other factors important for the design of the antisense nucleic acid include increasing the water solubility and cell membrane permeability and the like; these can also be achieved by improving dosage forms, such as the use of liposomes or microspheres.

Ribozymes capable of specifically cleaving the mRNA or initial transcription product that encodes the Jak1 or Jak1 signaling molecule within the coding region (in case of the initial transcription product, intron portion is included) can also be encompassed in the scope of antisense nucleic acids. “A ribozyme” refers to an RNA possessing an enzyme activity to cleave a nucleic acid. Since it has recently been shown that oligo-DNAs having a nucleotide sequence of the enzyme activity site also possess nucleic acid cleavage activity, however, this term is to be used herein as a concept encompassing DNA, as far as sequence-specific nucleic acid cleavage activity is possessed. The most versatile ribozymes are self-splicing RNAs found in infectious RNAs such as viroid and virusoid, and the hammerhead type, the hairpin type and the like are known. The hammerhead type exhibits enzyme activity with about 40 bases in length, and it is possible to specifically cleave the target mRNA alone by making several bases (about 10 bases in total) at both ends adjoining to the hammerhead structure portion to be a sequence complementary to the desired cleavage site of the mRNA. Furthermore, when the ribozyme is used in the form of an expression vector containing the DNA that encodes it, the ribozyme may be a hybrid ribozyme prepared by further joining a sequence modified from the tRNA to promote the migration of the transcription product to cytoplasm [Nucleic Acids Res., 29(13): 2780-2788 (2001)].

A double-stranded oligo-RNA having a nucleotide sequence complementary to a partial sequence within the coding region of the mRNA or initial transcription product that encodes the Jak1 or Jak1 signaling molecule (in case of the initial transcription product, intron portion is included) (siRNA) can also be encompassed in the scope of antisense nucleic acids. It had been known that what is called RNA interference (RNAi), the phenomenon where transfer of a short double-stranded RNA into a cell results in the degradation of mRNAs complementary to one strand of the RNA, occurs in nematodes, insects, plants and the like; since this phenomenon was confirmed to occur in animal cells as well (Nature, 411(6836), 494-498 (2001)), siRNA has been widely utilized as an alternative technique to ribozymes. The size of the siRNA is not subject to limitation, as far as RNAi can be induced; for example, the size can be 15 by or more, preferably 20 bp or more. An siRNA possessing RNAi activity can be prepared by separately synthesizing a sense strand and an antisense strand using an automated DNA/RNA synthesizer, and denaturing the strands in an appropriate annealing buffer solution at about 90° C. to about 95° C. for about 1 minute, and then performing annealing at, for example, about 30° C. to about 70° C., for about 1 to about 8 hours.

An expression vector capable of expressing a nucleic acid having a nucleotide sequence complementary to a nucleotide sequence that encodes the above-described Jak1 or Jak1 signaling molecule or a portion thereof is also preferable as a substance that inhibits a functional accentuation of the Jak1-related intracellular signaling system. The expression vector is preferably an expression vector capable of functioning in cells (T cells and the like) of the subject mammal, and can be provided in a state wherein a nucleic acid having a nucleotide sequence complementary to a nucleotide sequence that the encodes Jak1 or Jak1 signaling molecule or a portion thereof is operably joined downstream of an appropriate promoter [e.g., a promoter capable of exhibiting promoter activity in cells (T cells and the like) of the subject mammal] in the vector.

An antibody that specifically recognizes the Jak1 or Jak1 signaling molecule is capable of suppressing a function (e.g., interactions with other molecules, protein kinase activity and the like) of the Jak1 or Jak1 signaling molecule by binding to the Jak1 or Jak1 signaling molecule. The antibody may be a polyclonal antibody or a monoclonal antibody, and can be generated by a commonly known immunological technique. The antibody may also be a binding fragment of antibody [e.g., Fab, F(ab′)₂] or a recombinant antibody (e.g., single-chain antibody).

For example, the polyclonal antibody can be acquired by administering Jak1, a Jak1 signaling molecule or a fragment thereof as an antigen (as required, may be prepared as a complex crosslinked to a carrier protein such as bovine serum albumin or keyhole limpet hemocyanin), along with a commercially available adjuvant (e.g., Freund's complete or incomplete adjuvant), to an animal subcutaneously or intraperitoneally about 2 to 4 times at intervals of 2 to 3 weeks (the antibody titer of partially drawn serum has been determined by a known antigen-antibody reaction and its elevation has been confirmed in advance), collecting whole blood about 3 to about 10 days after final immunization, and purifying the antiserum. As the animal to receive the antigen, mammals such as rats, mice, rabbits, goat, guinea pigs, and hamsters can be mentioned.

A monoclonal antibody can be generated by a cell fusion method (e.g., Takeshi Watanabe, Saibou Yugouhou No Genri To Monokuronaru Koutai No Sakusei, edited by Akira Taniuchi and Toshitada Takahashi, “Monokuronaru Koutai To Gan—Kiso To Rinsho—”, pp. 2-14, Science Forum Shuppan, 1985). For example, the Jak1 or Jak1 signaling molecule or a fragment thereof, along with a commercially available adjuvant, is subcutaneously or intraperitoneally administered to mice 2 to 4 times; about 3 days after final administration, the spleen or lymph node is collected, and leukocytes are collected. These leukocytes and myeloma cells (e.g., NS-1, P3X63Ag8 and the like) are cell-fused to obtain a hybridoma that produces a monoclonal antibody that specifically recognizes the Jak1 or Jak1 signaling molecule. The cell fusion may be by the PEG method [J. Immunol. Methods, 81(2): 223-228 (1985)] or the voltage pulse method [Hybridoma, 7(6): 627-633 (1988)]. A hybridoma that produces a desired monoclonal antibody can be selected by detecting in the culture supernatant an antibody that binds specifically to an antigen using a well-known method of EIA or RIA and the like. Cultivation of a hybridoma that produces a monoclonal antibody can be achieved in vitro, or in vivo in the ascites fluid and the like of a mouse or a rat, preferably a mouse, and each antibody can be acquired from the culture supernatant of the hybridoma or the ascites fluid of the animal.

Taking into account the therapeutic efficacy and safety in humans, however, the above-described antibody may be a chimeric antibody or a humanized or human type antibody. A chimeric antibody can be prepared with reference to, for example, “Jikken Igaku (extra issue), Vol. 6, No. 10, 1988”, JP-B-3-73280 and the like. A humanized antibody can be prepared with reference to, for example, JP-A-4-506458, JP-A-62-296890 and the like. A human antibody can be prepared with reference to, for example, “Nature Genetics, Vol. 15, pp. 146-156, 1997”, “Nature Genetics, Vol. 7, pp. 13-21, 1994”, JP-A-4-504365, WO94/25585, “Nikkei Science, June issue, pp. 40-50, 1995”, “Nature, Vol. 368, pp. 856-859, 1994”, JP-A-6-500233 and the like.

Jak1 signaling inhibitor molecules and substances that enhance the expression or activity thereof can also be suitably included in the prophylactic and/or therapeutic drug for inflammatory skin disease of the present invention. Such substances include Jak1 signaling inhibitor molecules such as Socs3 as they are, nucleic acids that encode the same, expression vectors comprising the same and the like.

Nucleic acids that encode a Jak1 signaling inhibitor molecule such as Socs3 include cDNAs, mRNAs, chromosome DNAs and the like that encode a Jak1 signaling inhibitor molecule. The nucleic acid that encodes a Jak1 signaling inhibitor molecule sometimes has a varied sequence depending on the mammalian species and the like; such nucleic acids can also be used in the present invention. Although the nucleic acid may be a DNA or an RNA, or a DNA/RNA chimera, preference is given to a DNA. The nucleic acid may be double-stranded or single-stranded. In case of a double strand, the same may be a double-stranded DNA, a double-stranded RNA, or a DNA/RNA hybrid. A nucleic acid that encodes a Jak1 signaling inhibitor molecule can be prepared by amplification by a polymerase chain reaction (hereinafter abbreviated as “PCR method”) or reverse transcriptase-PCR (hereinafter abbreviated as “RT-PCR method”) using a synthetic primer comprising a portion of the nucleic acid and a template comprising a chromosome DNA, mRNA, cDNA or the like comprising the nucleic acid.

An expression vector comprising a nucleic acid that encodes a Jak1 signaling inhibitor molecule can be produced by operably joining the cloned nucleic acid downstream of a promoter in an appropriate expression vector. As the expression vector, an appropriate vector (plasmid vector, viral vector, retrovirus vector, baculovirus vector, adenovirus vector and the like) can be chosen according to the host used. For the promoter, an appropriate one can be chosen according to the host used.

Useful hosts include, for example, bacteria of the genus Escherichia (Escherichia coli and the like), bacteria of the genus Bacillus (Bacillus subtilis and the like), yeasts (Saccharomyces cerevisiae and the like), insect cells [established cells derived from fall armyworm larva (Spodoptera frugiperda cell; Sf cell) and the like], insects (silkworm larvae and the like), mammalian cells [simian cells (COS-7 and the like), Chinese hamster cells (CHO cells and the like) and the like] and the like.

An example of a Jak1 inhibitor as “a substance that inhibits a functional accentuation of the Jak1-related intracellular signaling system” is myricetin (3,3′,4′,5,5′,7-hexahydroxyflavone). Myricetin, a kind of flavonoid contained in various fruits and vegetables, is known to inhibit the phosphorylation of Jak1 and increase the self-phosphorylation of EGF receptor (EGFR) (Kumamoto T. et al., Cancer Lett. 2009, Mar. 8; 275(1): 17-26, Epub 2008 Nov. 7.). The substance is also commercially available (Nacalai Tesque and the like).

The amount of the active ingredient of the present invention blended in the prophylactic/therapeutic drug for inflammatory skin disease of the present invention is not particularly limited, as far as the desired effect is obtained; the amount can be chosen as appropriate over a broad range according to the choice of active ingredient used.

The dosage form of the prophylactic/therapeutic drug for inflammatory skin disease of the present invention is not particularly limited, as far as it can be applied to the skin; examples include a dosage form prepared by dissolving or mixing and dispersing the above-described active ingredient of the present invention in a base to obtain a cream, paste, jelly, gel, emulsion, liquid or other form (ointments, liniments, lotions and the like), a dosage form prepared by dissolving or mixing and dispersing the above-described active ingredient of the present invention in a base, and extending the solution or dispersion over a support (cataplasms and the like), a dosage form prepared by dissolving or mixing and dispersing the above-described active ingredient of the present invention in an adhesive, and extending the solution or dispersion over a support (plasters, tapes and the like) and the like.

Choice of the above-described base is not particularly limited, as far as it is pharmaceutically acceptable; conventionally known bases for ointments, liniments, lotions and the like can be used; examples include polymers such as sodium alginate, gelatin, cornstarch, tragacanth gum, methylcellulose, hydroxyethylcellulose, carboxymethylcellulose, xanthane gum, dextrin, carboxymethyl starch, polyvinyl alcohol, sodium polyacrylate, methoxyethylene-maleic anhydride copolymer, polyvinyl ether, and polyvinylpyrrolidone; oils and fats such as beeswax, olive oil, cacao oil, sesame oil, soybean oil, camellia oil, peanut oil, beef tallow, lard, and lanolin; white petrolatum, yellow petrolatum; paraffin; hydrocarbon gel ointments (e.g., Plastibase, manufactured by Bristol-Myers Squibb Company); higher fatty acids such as stearic acid; higher alcohols such as cetyl alcohol and stearyl alcohol; polyethylene glycol; water and the like.

The prophylactic/therapeutic drug for inflammatory skin disease of the present invention may be supplemented with carriers in common use added according to the dosage form thereof as required; examples of such carriers include diluents or excipients such as binders, surfactants, moisture retention agents, fillers, bulking agents, and wetting agents.

The prophylactic/therapeutic drug for inflammatory skin disease of the present invention can be prepared by a conventional method suitable for the dosage form thereof.

Provided that the substance that inhibits a functional accentuation of the Jak1-related intracellular signaling system is a nucleic acid, the agent of the present invention can further comprise a reagent for nucleic acid introduction to promote the introduction of the nucleic acid into cells. When the nucleic acid is incorporated in a virus vector, particularly in a retrovirus vector, retronectin, fibronectin, polybrene or the like can be used as a transfection reagent. When the nucleic acid is incorporated in a plasmid vector, an anionic lipid such as lipofectin, lipfectamine, DOGS (transfectam; dioctadecylamidoglycylspermine), DOPE (1,2-dioleoyl-sn-glycero-3-phosphoethanolamine), DOTAP (1,2-dioleoyl-3-trimethylammoniumpropane), DDAB (dimethyldioctadecylammonium bromide), DHDEAB (N,N-di-n-hexadecyl-N,N-dihydroxyethylammonium bromide), HDEAB (N-n-hexadecyl-N,N-dihydroxyethylammonium bromide), polybrene, or poly(ethyleneimine) (PEI) can be used.

Provided that the substance that inhibits a functional accentuation of the Jak1-related intracellular signaling system is a protein, the composition of the present invention can further comprise a reagent for polypeptide introduction to increase the efficiency of the introduction of the protein into cells. Useful reagents include Profect (Nacalai Tesque), ProVectin (IMGENEX) and the like.

The amount applied and the duration of application of the prophylactic and/or therapeutic drug for inflammatory skin disease of the present invention are set as appropriate according to the choice and activity of the active ingredient, the severity of the illness, the animal species to receive the drug, the drug tolerability, body weight, and age of the recipient, and the like. Normally, an effective amount of a dosage form that can be applied to the skin (e.g., ointments, liquids) is applied to the affected portion once a day or in several divided portions.

### (Method of Ameliorating a Functional Abnormality in the Skin Barrier)

The present invention provides a method of ameliorating a functional abnormality in the skin barrier. That is, the method is preferably carried out on the basis of an determination result obtained by the above-described method of the present invention for determining the presence or absence of a functional abnormality in the skin barrier.

Accordingly, the method of the present invention for ameliorating a functional abnormality in the skin barrier comprises:

(a) the step of measuring the expression of a protease and/or an inhibitor thereof on a skin surface in a test object,
(b) the step of measuring a serum IgE concentration, and
(c) the step of administering an effective amount of a substance that inhibits a functional accentuation of the Jak1-related intracellular signaling system to a test object that exhibited a change in the component balance of the protease and/or inhibitor thereof on the skin surface in the step (a), and did not exhibit a rise in the serum IgE concentration in the step (b).

The steps (a) and (b) are the same as the steps carried out in the above-described “method of determining the presence or absence of a functional abnormality in the skin barrier” of the present invention.

“A substance that inhibits a functional accentuation of the Jak1-related intracellular signaling system” used in the step (c) can be exemplified by the substances mentioned as examples included in the above-described prophylactic and/or therapeutic drug for inflammatory skin disease of the present invention.

Regarding the route of administration, transdermal administration is chosen since the invention of this application targets inflammatory skin diseases not due to an immunological abnormality, especially diseases due to a functional abnormality in the skin barrier, as described below, and also since the influences of the actions of the substance other than the desired action can be suppressed. It is particularly preferable that the prophylactic and/or therapeutic drug for inflammatory skin disease of the present invention be applied on the skin epidermis. The dosage form and the amount administered can be exemplified by those mentioned as examples of the above-described the prophylactic and/or therapeutic drug for inflammatory skin disease of the present invention.

“An effective amount” of a substance that inhibits a functional accentuation of the Jak1-related intracellular signaling system can be set as appropriate over a range that ensures an initial effect, on the basis of the choice and activity of the active ingredient, the severity of the illness, the animal species to receive the substance, the drug tolerability, body weight, and age of the recipient, and the like.

Furthermore, in the determination method of the present invention, by applying a skin protectant such as petrolatum to a topical site on the skin in a test object confirmed as having a functional abnormality in the skin, an inflammatory skin disease due to a functional abnormality in the skin barrier or a non-IgE-mediated inflammatory skin disease can be prevented and/or treated.

### (Diagnostic Kit for Inflammatory Skin Disease)

As stated above, in the event of a functional abnormality in the skin barrier and/or an inflammatory skin disease, accentuated expression of a series of proteases or a group of inhibitor molecules thereof is observed in the skin. Accordingly, the present invention provides a diagnostic kit for inflammatory skin disease comprising a substance capable of specifically detecting at least one kind of molecule out of these molecules.

These molecules have the expression levels varying widely upon development of atopic dermatitis in the above-described mouse model of inflammatory skin diseases, particularly atopic dermatitis. Specific examples are:

keratin 77;
Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;

### Lipocalin 2;

Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
ASldehyde oxidase 4;
Chitinase 3-like 1; and

### Keratin 16.

Preferably, it composed of the molecules having remarkably elevated expression levels. Specific examples include: Ubiquitin specific peptidase 18;

Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;

### Lipocalin 2;

Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and
Keratin 16, with greater preference given to:
Kallikrein related-peptidase 6,
Predicted gene, OTTMUSG00000000971,
WAP four-disulfide core domain 12,
Extracellular proteinase inhibitor,
Chitinase 3-like 1, and
Protease, serine 27 (marapsin).

A substance capable of specifically detecting at least one kind of molecule in the above-described group of molecules, which can be included in the diagnostic kit for inflammatory skin disease of the present invention is a substance capable of detecting the molecule at the gene or protein level; specific examples include substances possessing specific affinity for the gene for the molecule, and/or substances possessing specific affinity for the protein of the molecule.

Substances possessing specific affinity for the gene include probes and primer pairs which possess specific affinity for the gene; the specific affinity means the property of hybridizing to the desired gene only; therefore, the substance may be completely complementary to all or part of the gene, or may contain one to several mismatches, as far as the above-described features are ensured. The probe and primer pair are not particularly limited, as long as they have specific affinity for the gene; examples include oligo- or polynucleotides comprising all or part of the base sequence of the gene or a sequence complementary thereto and the like, and they are chosen as appropriate according to the form of the gene to be detected. The oligo- or polynucleotide is not subject to limitations with respect to the derivation thereof, as far as it possesses specific affinity for the gene, and it may be one purified by a conventional method from the necessary portion cleaved out from the gene. These oligo- or polynucleotides may be labeled with a fluorescent substance, an enzyme, a radioisotope or the like.

When analyzing the status of expression of a plurality of kinds at one time, reagents can also be prepared for use in microarrays.

For example, substances possessing specific affinity for the protein include, for example, an antibody possessing specific affinity for the protein or a fragment thereof; the specific affinity is the capability of specifically recognizing and binding to the protein by an antigen-antibody reaction. The antibody or fragment thereof is not particularly limited, as far as it is capable of specifically binding to the protein; the antibody or fragment thereof may be any one of a polyclonal antibody, a monoclonal antibody and a functional fragment thereof. These antibodies or functional fragments thereof are prepared using a method in common use in the art. For example, when using a polyclonal antibody, a method is available wherein an animal such as a mouse or a rabbit is immunized with the protein injected by subcutaneous administration in the back or by intraperitoneal or intravenous injection and the like, and an elevation of the antibody titer is waited, after which antiserum is collected. When using a monoclonal antibody, a method is available wherein a hybridoma is generated by a conventional method and the secretion therefrom is collected. To produce an antibody fragment, a method is commonly used wherein a cloned antibody gene fragment is allowed to be expressed by a microorganism and the like. The purity of the antibody, antibody fragment and the like is not particularly limited, as far as specific affinity for the protein is retained. These antibodies or fragments thereof may be labeled with a fluorescent substance, an enzyme, a radioisotope or the like.

Furthermore, commercial products may be used.

In addition to a substance capable of specifically detecting a molecule whose expression level changes widely upon development of skin inflammation, particularly a molecule whose expression level has rose, the kit of the present invention may comprise various reagents required for the detection which are packaged therein. Examples include buffer solutions, labeling reagents, reaction vessels and the like.

In a preferred embodiment, the diagnostic kit of the present invention comprises, for example, (i) an adhesive sheet for recovering a skin tissue sample from the test object, and (ii) a reagent for detecting and identifying a molecule contained in the skin tissue sample. The adhesive sheet has an adhesive force that preferably allows the recovery of only the layer containing a molecule whose expression is accentuated upon development of skin inflammation, without having a major impact on the skin. This adhesive force can be set as appropriate according to the material of the sheet used, application site, application period and the like. By examining the presence or absence of a molecule in the skin tissue sample recovered on the sheet using (ii) a reagent for detecting and identifying the molecule in the skin tissue sample, it is possible to diagnose whether the test object has contracted an inflammatory skin disease. By knowing the extent of the expression thereof, it is also possible to predict the seriousness and the degree of progression. The reagent comprises a substance possessing specific affinity for the above-described molecule (e.g., antibody).

### EXAMPLES

The present invention is hereinafter described in more detail with reference to the following examples, which, however, do not limit the scope of the present invention by any means. The reagents, apparatuses and materials used in the present invention are commercially available unless otherwise specified.

### (Methods and Materials)

### 1. Mice

A mouse model of atopic dermatitis was generated according to a detailed protocol reported [Masuya et al., Hum Mol Genet. 16, 2366-2375 (2007); http://www.brc.riken.jp/lab/gsc/mouse/]. C57BL/6J mice and C3H/HeJ mice were purchased from CLEA Japan. C57BL/6-kit^{W-sh/W-sh} mice were obtained from the RIKEN BioResource Center. C57BL/6-RAG1^{-/-} mice were allowed to propagate spontaneously. Severity scores were determined using an improved version of a reported scale [Matsuda et al., Int Immunol. 9, 461-466 (1997)]. The mice were reared in an SPF (specific pathogen-free) environment and subjected to experiments in compliance with the guidelines with the approval of RIKEN's Animal Experiment Committee.

In this model of dermatitis, dermatitis develops even in the SPF environment at 8 weeks after birth, and this is followed by an elevation of serum IgE level after 2 weeks. The specific time course is as follows.

1. Development of non-IgE-dependent dermatitis due to an abnormality in the skin barrier at around 8 weeks of age after birth
2. Transition to dermatitis with an immunological shift to the Th2 system, including elevated IgE and IgG1 levels and activation of mast cells and the like, with a delay of 2 weeks after the above-described development
3. Transition to chronic dermatitis with activation of the entire immune system, including the Th1 system, as indicated by elevated IgG2b and IgG2c levels at around 8 weeks after development of dermatitis

### 2. Mapping and Sequencing

A phenodeviant with a skin abnormality was mated with a wild-type C3H/HeJ mouse, and the resulting offspring were examined for phenotype transmission and the causal gene by gene mapping. A mapped single-nucleotide polymorphism (SNP) has been reported (Masuya et al., ibid., 2007). Out of candidate genes listed by PosMed (http://omicspace.riken.jp) search, the Jak1 gene was selected as the causal gene. Using a primer complementary to the Jak1 gene (available on demand), the gene was amplified by PCR and sequenced.

### 3. Western Blotting

Immunoprecipitation and immunoblotting were performed as described below.

Mouse embryonic fibroblasts (MEF cells) were cultured under serum-free conditions for 12 hours and stimulated with IL-6 (50 ng/ml). The cells were then lysed in lytic buffer (20 mM Tris-HCl [pH 7.4], 150 mM NaCl, 1% Nonidet P-40; supplemented with a protease inhibitors). The lysate was centrifuged (10,000×g, 30 minutes) and clarified. The clarified lysate, along with an anti-Jak1 antibody (BD Transduction Lab) or an anti-Stat3 antibody (Cell signaling) and protein A-Sepharose (GE Healthcare), was incubated for 4 hours. After the incubation, the immunoprecipitate was washed with a lytic buffer containing no protease inhibitors, separated by gradient SDS-PAGE, and transferred onto the PVDF Immobilon P membrane (Millipore). The membrane was incubated with each of an anti-pTyr antibody (4G10, Upstate), an anti-Jak1 antibody, an anti-pY-Stat3 antibody (Cell signaling) and an anti-Stat3 antibody (Cell signaling). After being washed with TBST (20 mM Tris-HCl [pH 7.4], 150 mM NaCl, 0.1% Tween 20), the membrane was immersed in a horseradish peroxidase-coupled goat anti-rabbit antibody (Zymed). The immunoconjugate was visualized using a chemiluminescence system (PerkinElmer Life Sciences. Inc.).

### 4. In Vitro Kinase Assay

Jak1 kinase activity was actually measured as reported [Fukada et al., Immunity 5, 449-60 (1996); Takahashi-Tezuka M. et al., Mol Cell Biol. 18, 4109-4117 (1998)]. 293T cells stably expressing a chimeric receptor composed of the extracellular domain of a granulocyte colony stimulating factor (G-CSF) receptor and the shortened intracellular domain of gp130 (293T-G133 cells) were transformed with an expression plasmid for mutant Jak1 (R878H) or wild-type Jak1, and then stimulated with G-CSF (100 ng/ml).

Ten minutes after the stimulation, HA-tagged Jak kinase was immunoprecipitated with an anti-HA antibody, washed with a lytic buffer containing no protease inhibitors, and immersed in a kinase buffer (60 mM HEPES [pH 7.3], 3 mM MgCl₂, 3 mM MnCl₂, 1.2 mM DTT, 1 mM ATP). The kinase reaction was carried out by incubation in a kinase buffer containing ATP (1 mM) and recombinant Stat3 protein (Active Motif) as base. The phosphorylation of the Stat3 and Jak1 proteins was analyzed by immunoblotting using an anti-pY-Stat3 antibody (Cell Signaling) and an anti-pY-Jak1 antibody (Cell Signaling), respectively.

### 5. Determination of Barrier Function

Trans-epidermal water loss (TEWL) was measured on the ventral and dorsal parts of the ear skin using the water loss measurement device, Tewameter TM 300 (Courage and Khazaka). Prior to measuring the permeability, an isotonic solution of NHS-LC-Biotin (Pierce) in PBS was coated to the ear skin. After incubation for 60 minutes, the ear skin was anatomized and frozen. Frozen sections were prepared, stained with Alexa546-coupled streptavidin (Invitrogen), and counter-stained with DAPI (Vector laboratories).

### 6. Generating Bone Marrow Chimeric Mice

Recipient mice (to be the host) were irradiated with a lethal dose of radiation from a ¹³⁷Cs source (9.5 Gy); 8 to 10 hours after the irradiation, bone marrow cells (1×10⁷ cells) from a different mouse (donor) were intravenously injected. The recipient mice were given drinking water containing 0.5 mg/ml neomycin sulfate and 0.3 mg/ml tetracycline hydrochloride (Calbiochem).

### 7. Topical Application of Jak Inhibitor

Jak inhibitor I (150 μM, pyridone 6; Calbiochem) in suspension in acetone/olive oil (4:1), 10 μl, was topically applied to the ventral and dorsal parts of the ear skin of each mutant mouse three times a week, starting at 4 weeks after birth.

### 8. ELISA

The blood was recovered from the submandibular vein, and EDTA-2K was added as an anticoagulant. Immunoglobulin and histamine levels in diluted plasma were measured using an ELISA kit (Bethyl and MBL) as directed in the instruction manual.

### 9. Proliferation Activity

Cytokine-dependent cell proliferation activity was measured as described below. Single cells of the thymus in suspension were cultured at a density of 5×10⁵ cells/well, and stimulated with PMA (10 ng/ml) [IL-2 (25 ng/ml), IL-4 (25 ng/ml), IL-6 (100 ng/ml), IL-7 (25 ng/ml), IL-12 (1-100 ng/ml) or IFN-γ (100 ng/ml) (Peprotech and R&D) was added]. Used in some experiments was the pan-Jak inhibitor Jak inhibitor I (3-100 nM, pyridone 6; Calbiochem) or Jak3 inhibitor I (125-500 μM, WHI-P131; Calbiochem). The cells were cultured for 48 hours, and their proliferation was measured by an assay using WST-8 (Nacalai Tesque) and soluble tetrazolium/formazan.

### 10. Histological and Histoimmunological Analyses

Ear tissue samples were prepared from mice, fixed in 4% para-formaldehyde/PBS, and embedded in polyester wax (VWR International); 5 μm thick sections were prepared and stained with hematoxylin-eosin or toluidine blue.

For histoimmunological analysis, frozen ear tissue specimens were fixed by microwave exposure for 10 seconds. All slide specimens were first incubated in 10% standard goat serum, 5% standard mouse serum in 3% BSA/PBS. Next, for detecting CD4, slide specimens were stained with biotin-conjugated anti-CD4 mAb (e-bio), then with horseradish peroxidase (HRP)-conjugated streptavidin. The reaction was visualized using the 3,3′-diaminobendidine (DAB) color developing agent (Dojin). When marapsin and chitinase 3-like 1 were to be detected, slide specimens were stained using an anti-marapsin antibody (goat; R&D) or an anti-chitinase 3-like 1 antibody (sheep; R&D), and then using a HRP-conjugated secondary antibody (anti-goat IgG or anti-sheep IgG; Zymed). The reaction was visualized using a DAB color developing agent (Dojin).

For analysis by immunofluorescence, frozen ear tissue specimens were fixed and stained using an antibody against pY-Jak1 (Cell Signaling), an antibody against Ki67 (Abcam), and an antibody against K6, K14 or K10 (Covance). Next, samples were incubated with an FITC- or Cy3-conjugated goat anti-rabbit IgG secondary antibody (Zymed). The slides were further counter-stained with hematoxylin or DAPI (Vector Laboratories).

### 11. Generating Mutant Jak1 Mice

A schematic diagram of the construction of a Jak1 mutant knock-in mouse is shown in FIG. 8.

A genomic clone carrying the mouse Jak1 gene, RP23-25A20, was obtained from a BAC clone collection of C57BL/6J mouse origin (Invitrogen). A 2.4 kbp BxtXI-Aor51HI DNA fragment and a 4.3 kbp Aor51HI-EcoRV DNA fragment, containing the exon 16-17 and exon 18-22 of the Jak1 gene, respectively, were subcloned into the pGEM-T Easy Vector (Promega) to yield targeting vectors. Next, a mis-sense mutation (R878H, a mutation from CGT to CAT) was introduced into the exon 19 of the Jak1 gene by recombination PCR technique. A targeting vector was constructed using the pMC1neoPolyA (Stratagene) plasmid as a backbone vector. To select products of homologous recombination, a loxp-Neo cassette was cloned into the Aor51HI site of the intron 17 of the Jak1 gene. To select products of random insertion, the thymidine kinase gene was inserted upstream of the 5′ arm. M1 ES cells were transfected with each linearized targeting vector by electroporation to yield mutant Jak1 mice. PCR and Southern blot analysis were performed on homologous recombinants using two ³²P-CTP-labeled DNA probes located outside of the targeting vector, and G-418- and ganciclovir-resistant clones were screened for. Specifically, the following primer sets were used for the PCR screening of the ES cells.

5′-side PCR amplification;
(SEQ ID NO: l)
5′-GAG TGG AGA AAG CTG ACC TGT G-3′,
and
(SEQ ID NO: 2)
5′-CGC CTT CTA TCG CCT TCT TGA C-3′;
3′-side PCR amplification;
(SEQ ID NO: 3)
5′-CAG TCA TAG CCG AAT AGC CTC-3′,
and
(SEQ ID NO: 4)
5′-AGC AGT CCA GAG GCT CAC AGC AAC-3′.

Southern blot analysis of ES cells was performed using the probes shown below. Each probe was prepared from a genomic DNA comprising the Jak1 gene by PCR amplification.

5′ probes (810 bp);
(SEQ ID NO: 5)
5′-CTA GCA TGA TGA GAC AGG TT-3′,
and
(SEQ ID NO: 6)
5′-TCC GGT GCA TGA AGA GAT CC-3′;
3′ probes (490 bp);
(SEQ ID NO: 7)
5′-CAG CAT GAA TGG CCA GAG TCC-3′,
and
(SEQ ID NO: 8)
5′-ATT GGT CCA TAA CAG CAC ATC-3′.

A mis-sense mutation in the (R878H) in the genomic DNA in the sorted ES cells was identified by DNA sequencing.

Germ-line chimeric mice were generated by the cell aggregation method. To generate a heterozygote of a mutant Jak1 mouse, a chimeric mouse having a high contribution of ES cells and a C57BL/6J mouse were mated.

### 12. Real-Time RT-PCR Analysis

Total cell mRNA was isolated from ear tissue using the RNeasy Gene Expression Kit (Qiagen) as directed in the instruction manual. RNA was reverse-transcribed using the High-capacity cDNA Reverse Transcription Kit (Applied Biosystems). For quantitative analysis of gene expression, real-time PCR was carried out using TaqMan Gene Expression Assays (Applied Biosystems).

### IL-4, Mm00445259_m1; IFN-γ, Mm00801778_m1; Jak1, Mm00600614_m1; Stat1, Mm00439518_m1; Stat3, Mm00456961_m1; Stat5a, Mm00839861_m1; Stat5b, Mm00839889_m1; and Stat6, Mm01160477_m1.

An amplification reaction was carried out on the ABI 7300 real-time PCR system using the TaqMan Gene Expression Master Mix (Applied Biosystems) with the cycle protocol of heat treatment at 95° C. for 10 minutes→at 95° C. for 15 seconds and at 60° C. for 1 minute in 40 cycles. Subsequently, Ct (threshold cycle) values were measured. Each Ct value indicates the number of cycles resulting in a specified amount amplified by the PCR. These values were standardized with the mouse ribosome housekeeping gene RPL19 (Mm02601633_g1) and calculated by the 2^{-ΔΔCt} method (here, ΔCt indicates the value obtained by subtracting the Ct value of RPL19 from the Ct value of the target gene, and ΔΔCt indicates the value obtained by subtracting the ΔCt value of the control from the ΔCt value of the sample).

### Example 1

### Identification of Mutation of the Causal Gene in Jak1 Tyrosine Kinase

The mouse model of atopic dermatitis generated as directed in the above section (Methods and Materials) was mated with a C3H/HeJ mouse, and a gene map was generated to identify the causal gene. Crossbreeding of a C3B6F1 heterozygote under SPF conditions resulted in the expression of the phenotype of dermatitis at a probability of ¼. Next, these two series were examined for phenotype transmission and the causal gene by gene mapping using SNP markers. As a result, the region that causes the illness was located in chromosome 4 (FIG. 1a). Chromosome 4 was divided, and some candidate genes were selected by PosMed search [Kobayashi N et al., Bioinformatics. 24:1002-10 (2008)]. A point mutation in the candidate gene Jak1 coding region was thus identified (FIG. 1b). This point mutation resulted in a change of the 878th arginine (R) located in the tyrosine kinase domain of Jak1 to histidine (H) (FIG. 1c). This mutation is hereinafter also referred to as R878H. Genome sequencing on all mice with the phenotype of dermatitis and their litter mates revealed a 100% agreement with the mutant phenotype, demonstrating that this Jak1 point mutation is homozygous (JAD/JAD mouse, also referred to as Spade/Spade mouse; Spade=Stepwise Progressive Atopic Dermatitis). The mutation was detected in the hetero-form in the hetero-mice. No such mutations were present in the wild-type phenotype.

As shown in FIG. 2, the consensus sequence in the Jak family comprises the underlined “tip of the tyrosine kinase domain” and also comprises an “ATP-binding consensus” (enclosed portion). The R878H mutation of Jak1 represents the replacement of the arginine upstream by two amino acids of the ATP-binding consensus by histidine, and has possibly influenced the ATP-binding activity. In Tyk2, the portion corresponding to the mutation point in Jak1 R878H is nearly the same sequence as of the Jak1 gene; by inducing the R898H mutation into Tyk2, it is possible to generate a functional accentuated Tyk2 molecule. In the Jak2 and Jak3 molecules, the portion corresponding to the 878th amino acid of Jak1 and the portion therearound are different amino acid sequences; however, by replacing the 851st to the 854th FLQQ in Jak2, or the 820th to 823rd YISL in Jak3, with RIRD to yield a chimeric molecule having the sequence of the wild-type Jak1 molecule, it is possible to replace the portion with an amino acid sequence nearly homologous to the sequence of Jak1 or Tyk2. Therefore, it is possible to generate a functional accentuated Jak2 molecule or Jak3 molecule by introducing RIHD in place of this RIRD.

To rule out the possibility that any unidentified mutation coexists in the vicinity of this point mutation in Jak1, a knock-in mouse having the same point mutation was generated by gene manipulation. The knock-in mouse was created by inserting the gene sequence constituted by the construct shown in FIG. 8 into the genome of C57BL/6 mouse ES cells by homologous recombination. ES cells successfully undergoing the homologous recombination were injected into blastocysts, and hetero-individuals were obtained from the resulting aggregation chimera. The hetero-individuals were mated to yield mice having the homo-, hetero-, or wild-type genes which were knockin genes. The mutant mouse homozygote is also referred to as the Jad/Jad mouse.

The knock-in mouse thus generated followed exactly the same pathogenetic course as with the previously reported ENU mutant mouse, including the development of dermatitis with severe itching at around 8 weeks of age, followed by a shift toward Th2 tendency showing elevated serum IgE and IgG1 levels after 2 weeks and the like. These findings confirmed that all cases of dermatitis developing in the ENU mutant mouse were due to this point mutation in the Jak1 gene.

### Example 2

### Functional Accentuation of Jak1 by Point Mutation

While some reports are available on mice with a mutation of the Jak1 gene, symptoms like dermatitis have not been reported as such mutations cause perinatal death in knock-out mice [Rodig et al., Cell. 93:373-83. (1998)]. Therefore, this point mutation in the Jak1 gene is estimated to give rise to a certain specific function that will induce dermatitis, rather than to cause a function loss. The point mutation in the nucleotide induces a single-amino-acid substitution known as R878H, and this was observed in the vicinity of the ATP-binding consensus domain that begins at the 880th amino acid within the tyrosine kinase domain of the Jak1 molecule (http://www.ncbi.nlm.nih.gov/Structure/cdd/wrpsb.cgi?seqinput=NP_{—}666257.2). The ATP-binding domain of tyrosine kinase is a sequence that is critical to the expression of tyrosine phosphorylation activity, and this consensus domain is conserved among the mouse and human Jak family proteins [Levine and Gilliland, Curr Opin Hematol. 14:43-7. (2007)]. Whether this point mutation influenced the tyrosine phosphorylation capability of this tyrosine kinase molecule was examined.

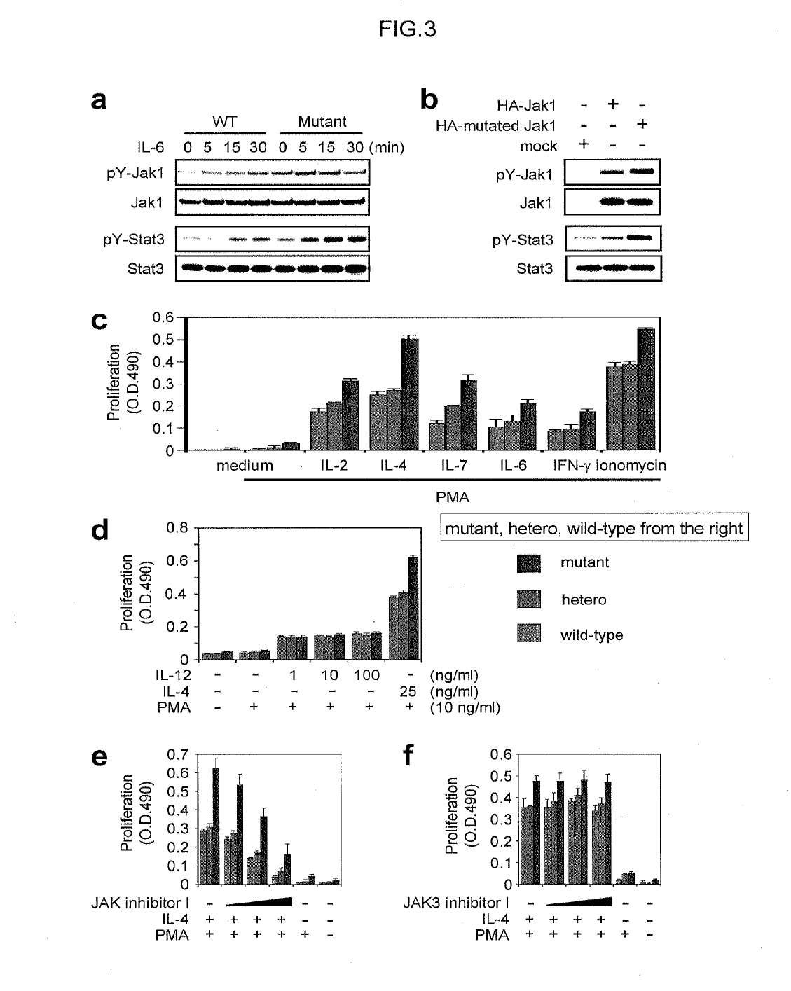

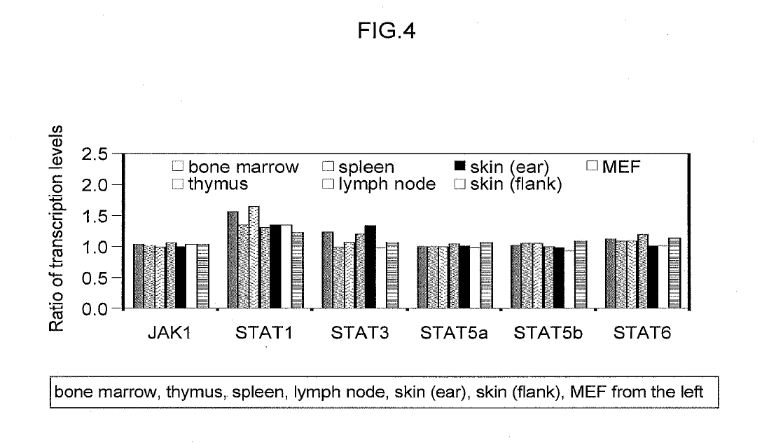

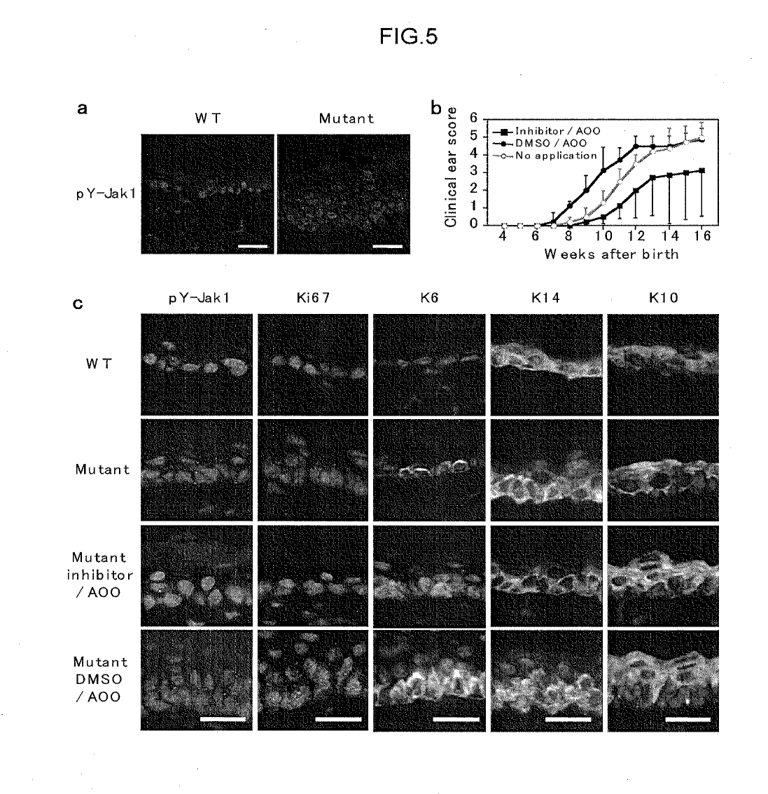

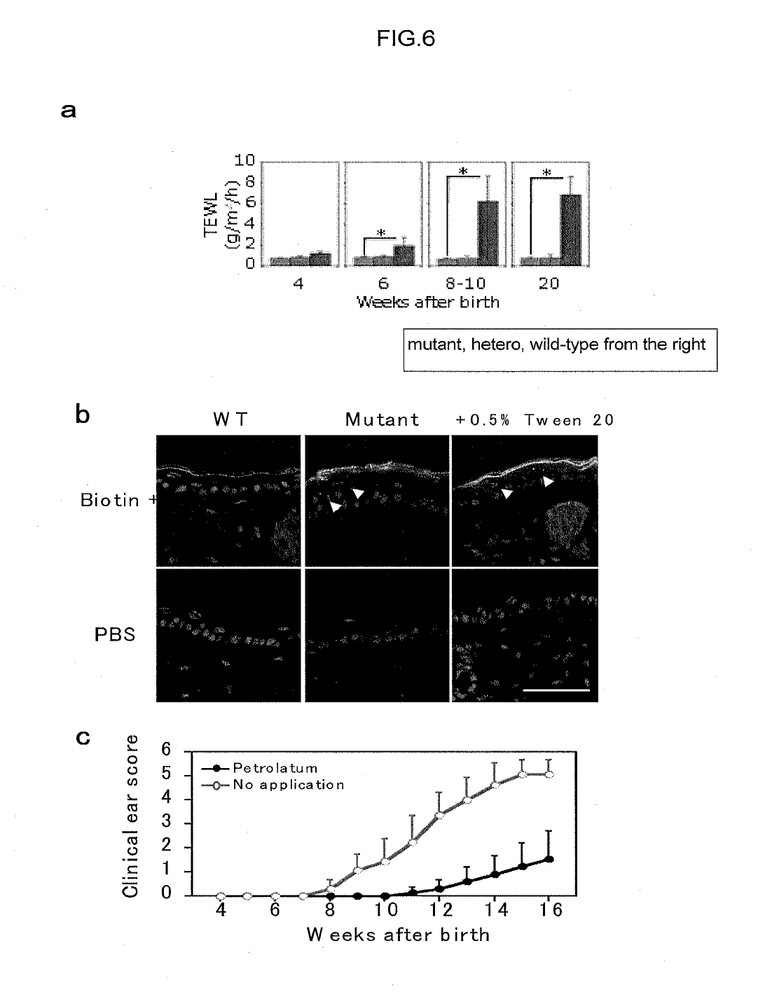

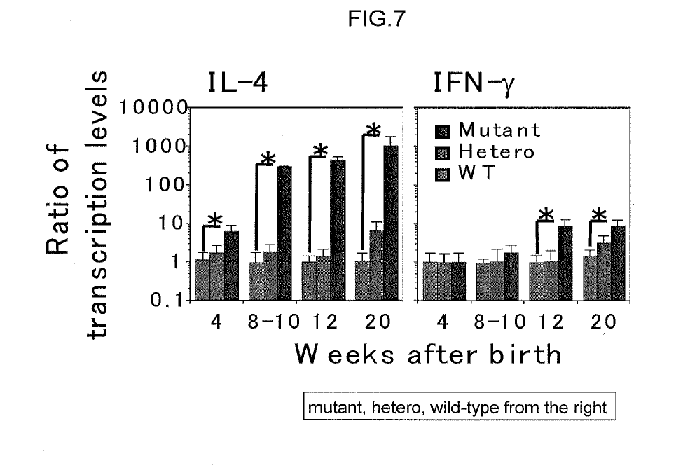

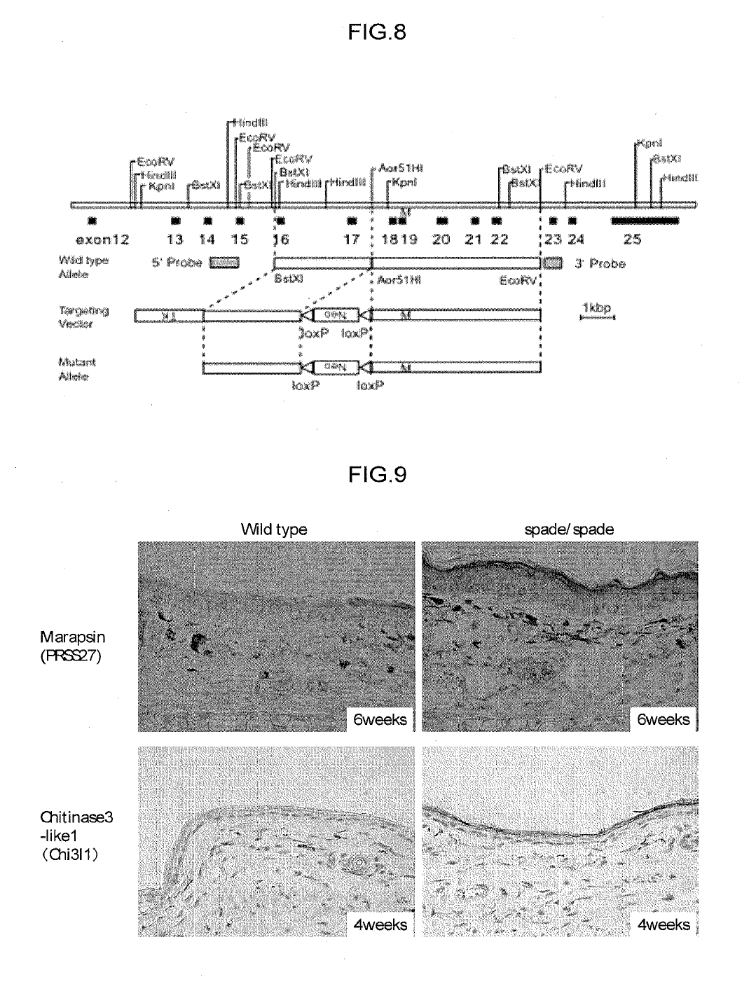

First, mouse embryonic fibroblast strains (MEF cells) were established from a mutant mouse (homozygote) and a wild-type mouse, and their MEF cells were compared in terms of the self-phosphorylation of Jak1 protein. Bothe before and after stimulation with the cytokine IL-6, the phosphorylation of Jak1 protein was accentuated in the MEF cells of the mutant mouse than in those of the wild-type mouse (two upper panels in FIG. 3a). In addition to the accentuation of the constitutive phosphorylation of Jak1 protein, an accentuation of the phosphorylation of Stat3 protein (a molecule involved in the signaling pathway located directly downstream of Jak1) was also noted (two lower panels in FIG. 3a). These results demonstrate excess activation of the tyrosine kinase function in the mutant Jak1 molecule.

Next, to determine whether the excess phosphorylation of Jak1 (mutant Jak1) in MEF cells derived from the mutant mouse is induced by excess activation of the tyrosine kinase activity of mutant Jak1, 293T cells expressing a G-CSFR-gp130 chimera receptor were transfected with HA-tagged mutant Jak1 or wild-type Jak1. After the cells were stimulated with G-CSF, HA-tagged mutant Jak1 or wild-type Jak1 protein was immunoprecipitated, and recombinant Stat3 phosphorylation was induced in vitro. Excess activation of mutant Jak1 protein was confirmed by strong tyrosine phosphorylation of recombinant Stat3 (FIG. 3b). These results demonstrate that this point mutation results in excess activation of the tyrosine phosphorylation potential of the Jak1 molecule.

### Example 3

### Influence of Jak1 Mutation on Excess Proliferation of Immunocytes

In mice with advanced dermatitis, the peripheral lymph nodes and spleen mostly hypertrophy, and the lymphocyte count increases. To examine the influence of Jak1 mutation on immunocyte proliferation, the in vitro proliferation potential of splenic CD4-positive T cells was compared between a mutant mouse (homozygote) and wild-type litter mates. Various cytokines that transmit their signals via Jak1 (e.g., IL-2, IL-4, IL-7, IL-6 and IFN-γ) induced excess proliferation of CD4-positive T cells derived from the mutant mouse (FIG. 3c); this excess proliferation was dose-dependently inhibited by the addition of the Jak inhibitor pyridone 6 (FIG. 3e), but not by the addition of the Jak3-specific inhibitor WHI-P131 (FIG. 3f). In contrast to these cytokines, when CD4-positive T cells were stimulated with IL-12 (which transmits a signal via Jak2, rather than via Jak1), no remarkable difference in cell proliferation was noted between the mutant mouse and the wild-type mice (FIG. 3d). These results showed that the R878H mutation in Jak1 induced the activation of Jak1-dependent signaling in immunocytes. However, the transcription levels of the mRNAs of Jak1 and various Stat were similar among the mutant, heterozygote and wild-type after stimulation with the cytokines (FIG. 4). This suggests that excess activation of Jak1 does not induce the transcriptional activity of these signaling substances.

### Example 4

### Induction of Dermatitis by Skin Environment

Jak1 has been reported to be involved in signaling pathways not only in immunocytes, but also in various series of cells such as skin epithelial cells and neurons [Crokera et al., Seminars in Cell & Developmental Biology 19: 414-422 (2008)]. To detect cells or tissues that cause the development of atopic dermatitis, bone marrow chimeric mice were generated from a combination of a wild-type mouse and a mutant mouse and examined for the development and progression of dermatitis (Table 1). Chimeric mice obtained by transplanting the bone marrow from the Jad/Jad mouse, the mutant mouse homozygote generated in Example 1, into a wild-type mouse host did not experience dermatitis even half a year after the transplantation. Chimeric mice obtained by transplanting the bone marrow from a wild-type mouse into a Jad/Jad mouse host experienced dermatitis at 10 weeks after the bone marrow transplantation. However, interestingly, the development of dermatitis after the bone marrow transplantation delayed by about 4 weeks, with less severe clinical symptoms, compared with the chimeric mice obtained with a combination of the Jad/Jad mouse host and the bone marrow from the Jad/Jad mouse. Dermatitis did not develop with a combination of the bone marrow from a wild-type mouse and a wild-type mouse host. These results led to the speculation that bone marrow cells may involve the exacerbation of dermatitis, but the cells (tissues) that contribute to the development of dermatitis may not bone marrow cells but skin tissue. To confirm the non-contribution of bone marrow cells, which are associated with various immunocytes, in the development of dermatitis, the Jad/Jad mouse was mated with an RAG1 knock-out mouse [Chen et al., Curr Opin Immunol. 6:313-9, (1994)] or a Kit^{Wsh} homozygote [Grimbaldeston et al., Am J. Pathol. 167:835-48, (2005)]. The RAG1 knock-out mouse (RAG1-NO mouse) is a mutant mouse lacking the formation of mature lymphocytes, whereas the Kit^{Wsh} homozygote is a mutant mouse lacking the formation of mast cells. In both RAG1-NO: Jak1 double mutant mouse and the Kit^{Wsh}: Jak1 double mutant mouse, dermatitis developed under SPF conditions (Table 2).

TABLE 1
Bone marrow transplantation experiment

| | Bone marrow | Dermatitis | Duration until | Maximum |
|---|---|---|---|---|
| Recipient mice | donor mouse | develop- | onset after | clinical |
| genotype | genotype | ment | transfer | ear score (6) |
| Wild-type | Wild-type | 0/8 | — | — |
| Wild-type | Mutant/Mutant | 0/8 | — | — |
| Mutant/Mutant | Wild-type | 10/10 | 11-19 weeks | 3.9 ± 0.5 |
| Mutant/Mutant | Mutant/Mutant | 8/8 | 5-18 weeks | 6.0 ± 0 |

TABLE 2
Genetic experiment

| | Dermatitis | Duration | Maximum |
|---|---|---|---|
| | develop- | until onset | clinical |
| Genotype | ment | after birth | ear score |
| JAK1^{R878H/R878H} | 8/8 | 8-11 weeks | 5.7 ± 0.4 |
| C57BL/6-RAG1⁺, | 8/8 | 9-12 weeks | 4.9 ± 0.5 |
| Mutant/Mutant | | | |
| C57BL/6-kit^{W-sh/W-sh}, | 8/8 | 8-14 weeks | 5.6 ± 1.1 |
| Mutant/Mutant | | | |

The fact of delayed development and decreased clinical scores under the RAG1-deficient conditions suggests that the functions of lymphocytes may influence the development and progression of dermatitis; these genetic studies have confirmed the contributions of skin tissue to the development of dermatitis.

### Example 5

### Destruction of Skin Barrier Permeability in Mutant Mice

First, to clarify the mechanism behind the development/progression of dermatitis in the skin of mutant mice, phosphorylated Jak1 protein (pY-Jak1) in the skins of a mutant mouse was analyzed. Remarkable accumulation of pY-Jak1 in the skin epidermal layer with dermatitis was confirmed, whereas the accumulation was rare in the skin epidermis of wild-type litter mates (FIG. 5a). Although pY-Jak1 was detected in the epidermis, it was not detected in the dermis. Accumulation of pY-Jak1 was noted in the skin epidermis of the mutant even before development of dermatitis (FIG. 5c), with some differentiation marker molecules expressed abnormally in the epidermal layer (FIG. 5c).

The strong expression of Ki67 in the ear skin of the mutant mouse indicates excess proliferation in epidermal layer cells. Expression of cytokeratin 6 in the skin of the mutant mouse, rather than in the skins of wild-type litter mates demonstrates that the differentiation of epidermal keratinocytes in the skin of the mutant mouse was in derangement. Expression of cytokeratin 14 is observed only in the basal membrane of the epidermis in the wild-type mice. In the skin of the mutant mice, however, the expression was observed in all layers; this demonstrates a deviation from normal differentiation. The high expression of cytokeratin 10 observed in the skin of the mutant mouse indicates the proliferation of epidermal keratinocytes in the mutant mouse epidermis. These results led to the notion that epidermal keratinocytes are contributory to the development of dermatitis in the mutant.

To confirm that a functional defect in the epidermal layer caused the dermatitis, the skin permeability barrier function in the mutant mouse was examined. Destruction of skin barrier permeability is a well-known phenomenon in atopic dermatitis patients [Cork et al., J Allergy Clin Immunol. 118:3-21, (2006); Bieber et al., N Engl J. Med. 358:1483-94, (2008)]. Calculating trans-epidermal water loss (TEWL) is a well-known method of determining a function of the skin barrier [Rosado et al., Int J Cosmet Sci. 27:237-41, (2005)]. TEWL from the ear skin of each of the mutant mouse homozygote and heterozygote and wild-type litter mates. The TEWL value began rising in the homozygote mice from before development of dermatitis at 4 weeks of age, and increased gradually. Regarding findings after development of dermatitis, both at 8 to 10 weeks, when development occurred, and at 20 weeks, when skin lichenization was seen, extremely high values of TEWL were obtained from the skins of the homozygotes (FIG. 6a).

In addition to the TEWL test to determine the skin barrier function of preventing water evaporation from the skin, the skin barrier function of preventing small molecules from infiltrating into the skin from outside was examined (FIG. 6b). A phosphate-buffered solution (PBS) containing biotin particles was applied to the ear skins of mice. The biotin particles readily infiltrated in the ear skins of mutant mouse homozygotes but did not infiltrate in the ear skins of wild-type mice. In FIG. 6b, the triangles indicate where the biotin particles deeply infiltrated in the skins of the homozygote mutant mice at the epidermal stratum spinosum. In contrast, biotin particles did not infiltrate the skin epidermis of the wild-type mice. When the surfactant Tween 20 was contained in the physiological saline, similar biotin infiltration was observed in the epidermis of the wild-type mice as well.

These results suggest that excess activation of mutant Jak1 causes abnormal genesis of epidermal keratinocytes and a resulting defect of skin barrier function.

### Example 6

### Influence of Jak1 Inhibitor on Dermatitis Development

The above-described Example has shown that dermatitis in the mutant mice is caused by excess activation of Jak1 and resulting defect of the skin barrier. To verify this hypothesis, a Jak inhibitor was coated on the ear skins of mutant mice at 4 weeks after birth and their litter mates. To allow the inhibitor to deeply penetrate the epidermal layer, a combination of acetone and olive oil was used as the solvent for the inhibitor. In fact, this solvent itself was harmful to the formation of the epidermal barrier; the development of dermatitis in the mutant mice was promoted by administering this solvent. By containing the Jak inhibitor, however, the development of dermatitis was delayed by 1 week, with less severe clinical symptoms, compared with a treatment with the solvent alone (FIG. 5b). In addition to preventing the development, coating with the Jak inhibitor suppressed the progression of the illness even after development of dermatitis. These findings show that the topical application of a Jak inhibitor is promising for medication that not only prevents the development of the illness, but also prevents the progression of the illness.

Since some differentiation marker molecules were abnormally expressed in the epidermal layer, how the expression of those differentiation marker molecules changed with inhibition of Jak1 activity with a Jak inhibitor in mutant mice was examined by histoimmunological staining (FIG. 5c). By inhibiting Jak1 activity, the expression of pY-Jak1 and the expression of Ki67 in the ear skins of the mutant mice could be reduced. In contrast, in the mice receiving the solvent alone, Jak1 and Ki67 were activated in excess. This demonstrates that topical application of a Jak inhibitor helps prevent the excess activation of Jak1. In the ear skins of mice treated with acetone-olive oil alone, all markers were expressed at higher levels than in the skins of non-treated mice. Nuclear staining of the epidermal cells with DAPI morphologically revealed activation of all epithelial cells.

### Example 7

### Influence of Petrolatum Coating on Development of Dermatitis

To determine whether the destruction of the skin permeability barrier causes the development of dermatitis in the mutant mice, their ear skins were coated with petrolatum alone three times a week for 4 to 16 weeks. Surprisingly, with this application of petrolatum to the ear skins, the development of dermatitis was delayed by 4 weeks on average, compared with non-treated mutant mice. Referring to the clinical score curves generated with mean values, the progression of the illness was slower in the non-treated group than in the group receiving the Jak inhibitor, although these systems included animals that did not experience the development of the illness at all before 16 weeks. Once dermatitis developed, the rising gradient of clinical scores agreed nearly completely to that of the control mice (FIG. 6c).

Interestingly, in animals that once began scratching their ears, petrolatum coating did not influence the progression of the illness at all. With the expectation for complete suppression of the development of the illness, a Jak inhibitor was added to petrolatum, but no additive effect was observed. The petrolatum treatment also inhibited infiltration by small particles. The observed complete prevention of the development of dermatitis by petrolatum coating demonstrates that the protection of the skin from water evaporation by petrolatum is beneficial in maintaining the skin barrier. This finding posed the possibility that even under SPF conditions, the development of early illness requires some extraneous stimulation through the destroyed skin barrier.

In relation to this possibility, the influence of the hygiene status of the animal breeding room was examined. The female Jad heterozygotes reared under SPF conditions did not experienced dermatitis during 1 year after birth, whereas some male mice experienced dermatitis when their skins were injured in fights (data not shown). This suggests that an unknown stimulation due to wound remodelling or infection with a certain microorganism plays a key role in the development of atopic dermatitis in the male heterozygote mice. To examine the influence of the hygiene status, the cages for the heterozygote mice were moved from the SPF clean room to a conventional breeding room. In five months after the movement, all heterozygote mice experienced atopic dermatitis. The above-described finding suggests that an external stimulation through the epidermis is in reality contributory to the development of dermatitis.

### Example 8

### Jak1-Mediated Signaling

To identify molecules becoming activated in mutant mice before development of dermatitis, mRNA was collected from mouse ear skins at 2 and 4 weeks after birth, before the development, and the expression of the mRNA was examined by microarray analysis or quantitative PCR analysis.

Samples of ear skins of mutant mouse homozygote, heterozygote and wild-type mice were examined for the mRNA expression of various cytokines (IL-2, IL-4, IL-5, IL-6, IL-7, IL-13, IFN-g, IL-17, IL-31 and TSLP). The expression levels of the Th2 cytokines IL-4 and IL-13 rose in the skin tissue before the development, whereas the expression level of the Th1 cytokine IFN-γ did not rise (FIG. 7).

### Example 9

### Microarray Analysis of Gene Expression Upon Development of Inflammatory Skin Disease

At 2, 4, and 6 weeks of age after birth, mRNA expression in ear tissues of atopic dermatitis mice and their litter mate wild-type mice was compared by microarray analysis. The genes whose expression levels differed by more than 2 times both at 4 and 6 weeks of age are listed in the table below.

| Genes whose expression levels | | | | |
|---|---|---|---|---|
| changed by a factor of 2 or more | | 2 weeks | 4 weeks | 6 weeks |
| (at 4 and 6 weeks of age) | | of age | of age | of age |
| 1433923_at | Keratin 77 | | 5.65 | 4.38 |
| 1436756_x_at | Hydroxyacyl- | | 2.59 | 2 |
| | Coenzyme A | | | |
| | dehydrogenase | | | |
| 1420570_x_at | T-cell leukemia/ | | 2.53 | 0.18 |
| | lymphoma 1B, 3 | | | |
| 1435541_at | Betacellulin, epidermal | 2.26 | 2.37 | 5.46 |
| | growth factor family | | | |
| | member | | | |
| 1418191_at | Ubiquitin specific | 0.35 | 0.48 | 0.47 |
| | peptidase 18 | | | |
| | Colony stimulating | | | |
| | factor 2 receptor, | | | |
| 1449360_at | beta 2, low-affinity | | 0.48 | 0.4 |
| | (granulocyte- | | | |
| | macrophage) | | | |
| 1417244_a_at | Interferon regulatory | | 0.47 | 0.42 |
| | factor 7 | | | |
| 1451375_at | Ets homologous factor | | 0.47 | 0.4 |
| 1417290_at | Leucine-rich alpha-2- | | 0.41 | 0.36 |
| | glycoprotein 1 | | | |
| 1434438_at | SAM domain and HD | | 0.46 | 0.39 |
| | domain, 1 | | | |
| 1427747_a_at | Lipocalin 2 | | 0.43 | 0.25 |
| 1429608_at | Alcohol dehydrogenase | | 0.43 | 0.24 |
| | 6A (class V) | | | |
| 1419598_at | Membrane-spanning | | 0.41 | 0.43 |
| | 4-domains, subfamily A, | | | |
| | member 6D | | | |
| 1420467_at | Psoriasis susceptibility | 0.33 | 0.41 | 0.31 |
| | 1 candidate 2 (human) | | | |
| 1449475_at | ATPase, H+/K+ | 0.39 | 0.39 | 0.39 |
| | transporting, nongastric, | | | |
| | alpha polypeptide | | | |
| 1449902_at | Late comified envelope | | 0.38 | 0.27 |
| | 1F | | | |
| 1454632_at | RIKEN cDNA | | 0.36 | 0.21 |
| | 6330442E10 gene | | | |
| 1419394_s_at | S100 calcium binding | | 0.36 | 0.09 |
| | protein A8 (calgranulin | | | |
| | A) | | | |
| | Solute carrier family 5 | | | |
| 1455431_at | (sodium/glucose | | 0.35 | 0.27 |
| | cotransporter), member 1 | | | |
| 1436530_at | Predicted gene, | | 0.35 | 0.13 |
| | OTTMUSG00000000971 | | | |
| 1418580_at | Receptor transporter | 0.3 | 0.33 | 0.22 |
| | protein 4 | | | |
| 1449420_at | Phosphodiesterase 1B, | 0.29 | 0.3 | 0.3 |
| | Ca2+-calmodulin | | | |
| | dependent | | | |
| 1449191_at | WAP four-disulfide | 0.4 | 0.3 | 0.11 |
| | core domain 12 | | | |
| 1448982_at | Kallikrein related- | 0.42 | 0.26 | 0.1 |
| | peptidase 6 | | | |
| 1417160_s_at | Extracellular proteinase | 0.29 | 0.25 | 0.1 |
| | inhibitor | | | |
| 1440686_at | Protease, serine 27 | | 0.25 | 0.17 |
| 1419215_at | Aldehyde oxidase 4 | 0.38 | 0.23 | 0.11 |
| 1451537_at | Chitinase 3-like 1 | 0.16 | 0.17 | 0.09 |
| 1448932_at | Keratin 16 | 0.23 | 0.15 | 0.02 |

The numerical figures on the three rows on the right side of the table were calculated by dividing the amounts of mRNA expressed in the skins of the wild-type mice by the amounts of mRNA expressed in the skins of the mutant mouse homozygotes experiencing atopic dermatitis at 2, 4 and 6 weeks of age. A value exceeding 2 indicates a reduction in the amount expressed in the mutant mice; a value under 0.5 indicates an accentuation of the amount expressed in the mutant mice. For the values as of age 2 weeks, only the genes whose expression levels changes by a factor of 2 times are shown.

Furthermore, increased expression of Proteinase, serine 27 (marapsin) on the skin surface was observed even at the protein level (FIG. 9).

The following 11 genes are known to increase their expression for at least 1 month before development, from 2 to 4 and 6 weeks of age.

Ubiquitin specific peptidase 18
Psoriasis susceptibility 1 candidate 2 (human)
ATPase, H+/K+ transporting, nongastric, alpha polypeptide
Receptor transporter protein 4
Phosphodiesterase 1B, Ca2+-calmodulin dependent
WAP four-disulfide core domain 12
Kallikrein related-peptidase 6
Extracellular proteinase inhibitor
Aldehyde oxidase 4
Chitinase 3-like 1

### Keratin 16

Because kallikrein related-peptidase 6 (KLK6) belongs to the same family as with KLK7 (also known as SCCE), which is known to mediate the turnover of the keratinous layer by decomposing and desquamating old skins, and also because its expression in the skin has been confirmed, it can be judged that KLK6 may also be involved in the turnover of the keratinous layer. Molecules that are functionally associated therewith include the predicted gene OTTMUSG00000000971, WAP four-disulfide core domain 12, extracellular proteinase inhibitor, and protease, serine 27.

In the skins of the mutant mice, from 2 weeks to 6 weeks of age, the amount of chitinase 3-like 1 molecule expressed was accentuated (FIG. 9). This is a substance structurally similar to chitinase, an enzyme for capturing and decomposing chitin as foreign matter from bacteria, hypha, insect coats and the like. Like chitinase, it is capable of binding to chitin but lacks the capability of decomposing the same. High expression of this molecule in the skins of the mutant mice was confirmed by histoimmunochemistry, suggesting the importance of this molecule in the development of the illness. An event that suggests the importance of this molecule is the change in the incidence in the hetero-mice depending on the rearing environment. By rearing the hetero-mice in an environment of low cleanliness, atopic dermatitis can be induced to develop; this can be thought to suggest the involvement of some environmental factors (e.g., bacterial infection) in addition to destruction of the skin barrier. The chitinase 3-like 1 molecule exhibits increased expression in the airway epithelium in asthma patients; SNP of the gene has been suggested to produce linkage disequilibrium in allergy patients; its expression is accentuated in the intestinal epithelium in patients with ulcerative colitis. Furthermore, in vitro experiments have shown that expressing this molecule in intestinal epithelial cells accentuates the uptake of infectious bacteria in epithelium.

Judging from these findings, it can be conjectured that the accentuated expression of chitinase 3-like 1 in the mutant mice is effective in inducing dermatitis to develop due to bacterial infection or uptake of an allergen such as house dust at the time of development of allergic disease.

Atopic dermatitis mice repeat scratching behavior after development of dermatitis. If the skin barrier is destroyed due to this mechanical stimulation, the expression of protease and an inhibitor thereof is accentuated for tissue regeneration. It is suggested that a derangement of the expressional balance among a plurality of proteases and inhibitors thereof in the epidermis of atopic dermatitis mice activates Protease-Activated Receptor (PAR2), and that this activation accentuates the production of cytokines such as TSLP (thymic stromal lymphopoietin) which may act on the activation of the Th2 immune system.

### Example 10

### Induction of Development of Psoriasis-Like Dermatitis by Deletion of Socs3 Specifically in Epidermal Cells

Some systems are present in living organism for providing suppressive control of the activation pathway by the Jak/Stat signaling system, the suppressor of cytokine signaling (SOCS) being a representative thereof. Socs molecules constitute their own family, a member of which, Socs3, is known to suppressively control signaling from Jak1. With this in mind, the K5creTgX Socs 3 f/f mouse (Socs3-deficient mouse) was generated by specifically deleting this gene from keratinized cells.

Specifically, to delete the expression of the SOCS3 gene in the skin, a knock-in mouse having a genome sequence with the SOCS3 gene inserted between loxp sequences, created by homologous recombination, was generated, and this was mated with the transgenic mouse K5CreTG, which expresses Cre specifically in the skin, whereby the region sandwiched by the loxp sequences was successfully deleted selectively in the skin. Thus, a skin-specific Socs3-deficient mouse was generated.

The Socs3-deficient mouse began experiencing dermatitis accompanied by scratching behavior at 3 months of age after birth, and more than 90% of the animals experienced severe dermatitis by 5 months of age after birth. All these mice with dermatitis exhibited a rise in IgE antibody titer but none of them had an increase in mast cells or degranulation at dermatitis sites, which are characteristically observed in atopic dermatitis, so that they were thought to be of a type of dermatitis different from atopic dermatitis. Neutrophil infiltration and severe vasodilation were noted in the lesions of the Socs3-deficient mouse, the dermatitis being pathologically like psoriasis.

Two models of scabies have been proposed to date: the IL-23-Stat3-Th17-IL-22-Defensine/antibacterial protein S100A7 pathway, which is mediated by Th17 cells, and the IL-23-Stat3-IL-19/IL-20/IL-24-Defensine/antibacterial protein S100A8/A9 pathway, which is not dependent on T cells. However, scabies-like dermatitis in the Socs3-deficient mice was found to be under the control of the IL-6-Stat3-IL-19/IL-24-Defensine and S100A8/A9 pathway, which originates from IL-6, and to form the psoriasis-like condition via a pathway distinct from that for conventional models.

This finding shows that Socs3 is functionally important in that it suppressively controls the activation of Stat3 by IL-6, which involves the proliferation of keratin cells to suppress the abnormal growth of the skin, thus maintaining homeostasis. Collapse of this suppressing mechanism induces abnormal proliferation of corneocytes, leading to the development of psoriasis-like dermatitis. This finding revealed a new aspect of Socs3 in skin homeostasis.

In the Socs3-deficient mice, destruction of the skin barrier by shaving cream induced keratinous layer thickening and accentuated the expression of IL-19, IL-24, Defensine, and S100A8/A9, which led to the induction of dermatitis. As such, these changes induced by this destruction of the skin barrier were resistant to anti-histamines, susceptible to FK506, and susceptible to Jak inhibitors.

The Socs3-deficient mice (a mouse model of psoriasis) had aspects very similar to those of the model of atopic dermatitis, including high IgE antibody titers and production of IL-4 and IL-13 in dermatitis sites as observed by transcription level analysis. This elevation of IgE antibody was observed always after development of dermatitis as with the Jak1 mutant mouse homozygote. This suggested destruction of the skin barrier led to an elevation of IgE antibody as in the mutant mouse homozygote having a Jak1 mutation.

### INDUSTRIAL APPLICABILITY

Based on the elucidation of the pathogenetic mechanism for the onset of inflammatory skin disease at the molecular and cellular levels, the invention of this application makes it possible to genetically diagnose the development of an inflammatory disease such as atopic dermatitis, and to develop a method of predicting the development, a method of preventing the development, and a method of treatment after the development.

This application is based on a patent application No. 2009-215035 filed in Japan, the contents of which are incorporated in full herein.

### SEQUENCE LISTING FREE TEXT

SEQ ID NO:1: 5′ PCT primer
SEQ ID NO:2: 5′ PCT primer
SEQ ID NO:3: 3′ PCT primer
SEQ ID NO:4: 3′ PCT primer
SEQ ID NO:5: 5′ probe
SEQ ID NO:6: 5′ probe
SEQ ID NO:7: 3′ probe
SEQ ID NO:8: 3′ probe

1. A method of determining the presence or absence of a functional abnormality in the skin barrier, comprising the step of measuring the expression of a protease and/or an inhibitor thereof on a skin surface in a test object.

2. The method according to claim 1, wherein the method comprises the step of collecting serum from the test object and determining the IgE concentration in the serum, and further comprises the step of differentially diagnosing whether IgE mediates the functional abnormality in the skin barrier in the test object.

3. The method according to claim 2, wherein the protease and/or inhibitor thereof is at least one kind selected from the group consisting of:
Keratin 77;
Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;
Lipocalin 2;
Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and
Keratin 16.

4. The method according to claim 2, wherein the protease and/or inhibitor thereof is at least one kind selected from the group consisting of:
Predicted gene, OTTMUSG00000000971;
WAP four-disulfide core domain 12;
Kallikrein related-peptidase 6;
Extracellular proteinase inhibitor; and
Protease, serine 27.

5. The method according to claim 2, wherein the method further comprises the step of measuring the presence or absence of a functional accentuation of the Jak1-related intracellular signaling system in the test object.

6. The method according to claim 5, wherein the functional accentuation of the Jak1-related intracellular signaling system is an accentuation of Jak1 activity, an accentuation of Stat3 activity, and/or a reduction in Socs3 activity.

7. The method according to claim 2, wherein the test object has developed or is likely to develop skin inflammation.

8. The method according to claim 7, wherein the method comprises determining the presence or absence of a functional abnormality in the skin barrier in a test object differentially diagnosed to be not mediated by IgE in the step of measuring the IgE concentration in the serum.

9. The method according to claim 8, wherein the method comprises using a skin tissue sample from the test object.

10. A diagnostic kit for a functional abnormality in the skin barrier, comprising a substance capable of specifically detecting at least one kind selected from the group consisting of:
Keratin 77;
Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;
Lipocalin 2;
Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and
Keratin 16.

11. The kit according to claim 10, wherein the kit comprises a substance capable of specifically detecting at least one kind selected from the group consisting of:
Predicted gene, OTTMUSG00000000971;
WAP four-disulfide core domain 12;
Kallikrein related-peptidase 6;
Extracellular proteinase inhibitor; and
Protease, serine 27.

12. The kit according to claim 11, wherein the kit further comprises a material having a level of adhesive force for collecting a skin tissue sample.

13. A prophylactic and/or therapeutic drug for skin inflammatory disease comprising a substance that inhibits a functional accentuation of the Jak1-mediated intracellular signaling system, wherein the drug is to be applied to the skin.

14. The drug according to claim 13, wherein the substance that inhibits a functional accentuation of the Jak1-mediated intracellular signaling system is a Jak1 inhibitor, Socs3, and/or a Stat3 inhibitor.

15. A method of ameliorating a functional abnormality in the skin barrier, comprising (a) the step of measuring the expression of a protease and/or an inhibitor thereof on a skin surface in a test object, (b) the step of measuring a serum IgE concentration, and (c) the step of administering an effective amount of a substance that inhibits a functional accentuation of the Jak1-related intracellular signaling system to a test object that exhibited a change in the component balance of the protease and/or inhibitor thereof on the skin surface in the step (a), and did not exhibit a rise in the serum IgE concentration in the step (b).

16. The method according to claim 15, wherein the protease and/or inhibitor thereof is at least one kind selected from the group consisting of:
Keratin 77;
Hydroxyacyl-coenzyme A dehydrogenase;
T-cell leukemia/lymphoma 1B, 3;
Betacellulin, epidermal growth factor family member;
Ubiquitin specific peptidase 18;
Colony stimulating factor 2 receptor, beta 2, low-affinity (granulocyte-macrophage);
Interferon regulatory factor 7;
Ets homologous factor;
Leucine-rich alpha-2-glycoprotein 1;
SAM domain and HD domain, 1;
Lipocalin 2;
Alcohol dehydrogenase 6A (class V);
Membrane-spanning 4-domains, subfamily A, member 6D;
Psoriasis susceptibility 1 candidate 2 (human);
ATPase, H+/K+ transporting, nongastric, alpha polypeptide;
Late cornified envelope 1F;
RIKEN cDNA 6330442E10 gene;
S100 calcium binding protein A8 (calgranulin A);
Solute carrier family 5 (sodium/glucose cotransporter), member 1;
Predicted gene, OTTMUSG00000000971;
Receptor transporter protein 4;
Phosphodiesterase 1B, Ca2+-calmodulin dependent;
WAP four-disulfide core domain 12;
Kallikrein related-peptidase 6;
Extracellular proteinase inhibitor;
Protease, serine 27 (marapsin);
Aldehyde oxidase 4;
Chitinase 3-like 1; and
Keratin 16.

17. The method according to claim 15, wherein the protease and/or inhibitor thereof is at least one kind selected from the group consisting of:
Predicted gene, OTTMUSG00000000971;
WAP four-disulfide core domain 12;
Kallikrein related-peptidase 6;
Extracellular proteinase inhibitor; and
Protease, serine 27.

18. The method according to claim 15, wherein the test object has developed or is likely to develop skin inflammation.

19. The method according to claim 15, wherein the skin inflammation is of the non-IgE-mediated type.

20. The method according to claim 15, wherein a skin tissue sample from the test object is used in the step (a).

21. The method according to claim 15, wherein the substance that inhibits a functional accentuation of the Jak1-related intracellular signaling system is a Jak1 inhibitor, Socs3, and/or a Stat3 inhibitor.
